(19) United States
(12) Patent Application Publication
(10) Pub. No.: US 2006/0122232 A1
(43) Pub. Date: Jun. 8, 2006

(54) 4-Hydroxyfuroic acid derivatives

(75) Inventors: Shan-Yen Chou, Taipei (TW); Shieh-Shung T. Chen, Morganville, NJ (US); Henry J. Tsai, Palos Verdes Estates, CA (US)

Correspondence Address: FISH & RICHARDSON PC, P.O. BOX 1022, MINNEAPOLIS, MN, 55440-1022, US

(73) Assignee: DEVELOPMENT CENTER FOR BIOTECHNOLOGY

(21) Appl. No.: 11/293,981

(22) Filed: Dec. 5, 2005

Related U.S. Application Data
(60) Provisional application No. 60/633,492, filed on Dec. 6, 2004.

(51) Int. Cl. A61K 31/4439; A61K 31/404; C07D 405/14

(52) U.S. Cl. 514/337; 514/414; 546/278.1; 548/465

(57) ABSTRACT

This invention relates to treating diabetes mellitus with certain 4-hydroxyfuroic acid derivatives. These derivates are of formula (I) below. Each variable is defined in the specification.

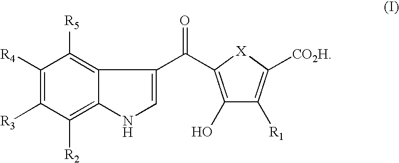

### CROSS REFERENCE TO RELATED APPLICATION

This application claims priority to U.S. Provisional Application Ser. No. 60/633,492, filed on Dec. 6, 2004, the contents of which are incorporated herein by reference.

### BACKGROUND

Type I diabetes or Type II diabetes are two major types of diabetes mellitus. Type I diabetic patients have low plasma insulin levels due to a lack of insulin produced by the beta cells of the pancreas. On the other hand, type II diabetic patients typically have normal plasma insulin levels, but are not responsive to the glucose and lipid metabolism in major insulin-sensitive tissues, such as muscle and liver.

One way to treat type I or type II diabetes is administration of exogenous insulin supplements. As insulin is a peptide not readily absorbed through the gastrointestinal tract, it is generally administered via tedious and painful subcutaneous injection.

There is a need to develop an insulin replacement that can be administered orally.

### SUMMARY

This invention is based on the discovery that certain 4-hydroxyfuroic acid derivatives activate insulin receptors and can be used as a insulin replacement to treat diabetes mellitus (e.g., type I diabetes or type II diabetes).

In one aspect, this invention features a compound of formula (I): 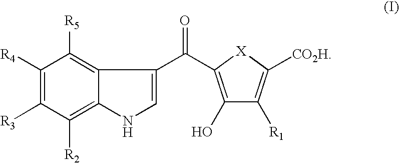
In this formula, X is O; R₁ is H, ORₐ, C₁-C₁₀ alkyl, C₃-C₂₀ cycloalkyl, C₃-C₂₀ heterocycloalkyl, aryl, 5-membered heteroaryl, 6-membered heteroaryl, or fused heteroaryl optionally substituted with C₁-C₄ alkyl, C₃-C₇ cycloalkyl, C₃-C₇ heterocycloalkyl, aryl, heteroaryl, halogen, or ORₐ; R₂ is C₁-C₁₀ alkyl optionally substituted with OR_{b}, COOR_{b}, C(O)NR_{b}R_{c}, or NR_{b}—C(O)R_{c}; each of R₃, R₄, and R₅, independently, is H, OR_{d}, halogen, C₁-C₁₀ alkyl, C₃-C₂₀ cycloalkyl, C₃-C₂₀ heterocycloalkyl, aryl, or heteroaryl; in which each of Rₐ, R_{b}, R_{c}, and R_{d}, independently, is H, C₁-C₁₀ alkyl, C₃-C₂₀ cycloalkyl, C₃-C₂₀ heterocycloalkyl, aryl, or heteroaryl.

Referring to formula (I), a subset of the just-described compounds are those in which R₁ is 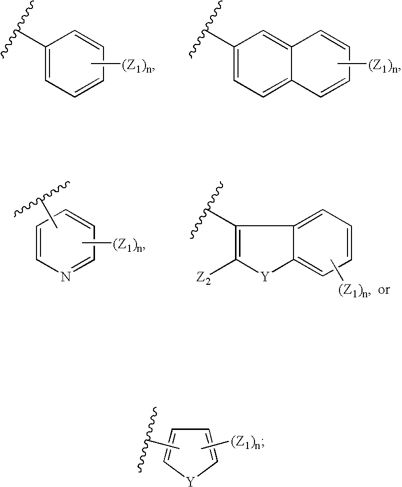
in which Y is O, S, N(R); n is 0-3; and each of Z₁ and Z₂, independently, is C₁-C₄ alkyl, C₃-C₇ cycloalkyl, C₃-C₇ heterocycloalkyl, aryl, heteroaryl, halogen, or OR′; each of R and R′, independently, being C₁-C₄ alkyl, C₃-C₇ cycloalkyl, C₃-C₇ heterocycloalkyl, aryl, or heteroaryl. In these compounds, R₂ can be C₁-C₁₀ alkyl optionally substituted with OR_{b} or COOR_{b}.

The term “alkyl” refers to a saturated or unsaturated, linear or branched, non-aromatic hydrocarbon moiety, such as —CH₃, —CH₂—, —CH₂—CH═CH₂—, or branched —C₃H₇. The term “cycloalkyl” refers to a saturated or unsaturated, non-aromatic, cyclic hydrocarbon moiety, such as cyclohexyl or cyclohexen-3-yl. The term “heterocycloalkyl” refers to a saturated or unsaturated, non-aromatic, cyclic moiety having at least one ring heteroatom, such as 4-tetrahydropyranyl or 4-pyranyl. The term “aryl” refers to a hydrocarbon moiety having one or more aromatic rings. Examples of an aryl moiety include phenyl, phenylene, naphthyl, naphthylene, pyrenyl, anthryl, and phenanthryl. The term “heteroaryl” refers to a moiety having one or more aromatic rings that contain at least one heteroatom. Examples of a heteroaryl moiety include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl. The term “4-hydroxyfuroic acid derivatives” includes both furoic acid derivatives and thienoic acid derivatives.

Alkyl, cycloalkyl, heterocycloalkyl, aryl, and heteroaryl mentioned herein include both substituted and unsubstituted moieties, unless specified otherwise. Possible substituents on cycloalkyl, heterocycloalkyl, aryl, and heteroaryl include C₁₋₁₀ alkyl, C₂-C₁₀ alkenyl, C₂-C₁₀ alkynyl, C₃-C₈ cycloalkyl, C₅-C₈ cycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, hydroxyl, halogen, thio, C₁-C₁₀ alkylthio, arylthio, C₁-C₁₀ alkylsulfonyl, arylsulfonyl, cyano, nitro, acyl, acyloxy, carboxyl, and carboxylic ester. On the other hand, possible substituents on alkyl, alkoxy, alkylthio, alkylamino, and dialkylamino include all of the above-recited substituents except C₁-C₁₀ alkyl, C₂-C₁₀ alkenyl, and C₂-C₁₀ alkynyl. Cycloalkyl and heterocycloalkyl can also be fused with aryl or heteroaryl.

In another aspect, this invention features a compound of formula (I) shown above except that X is S; R₁ is H, C₁-C₁₀ alkyl, C₃-C₂₀ cycloalkyl, C₃-C₂₀ heterocycloalkyl, aryl, heteroaryl, or ORₐ; and R₂ is unsubstituted C₁-C₁₀ alkyl or C₁-C₁₀ alkyl substituted with OR_{b}, COOR_{b}, C(O)NR_{b}R_{c}, or NR_{b}—C(O)R_{c}. Referring to formula (I), a subset of the just-described compounds are those in which R₁ is 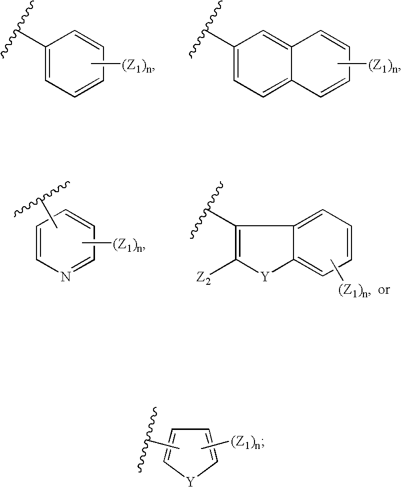
in which Y is O, S, N(R); n is 0-3; and each of Z₁ and Z₂, independently, is C₁-C₄ alkyl, C₃-C₇ cycloalkyl, C₃-C₇ heterocycloalkyl, aryl, heteroaryl, halogen, or OR′; each of R and R′, independently, being C₁-C₄ alkyl, C₃-C₇ cycloalkyl, C₃-C₇ heterocycloalkyl, aryl, or heteroaryl. In these compounds, R₂ can be C₁-C₁₀ alkyl substituted with OR_{b} or COOR_{b}.

In still another aspect, this invention features a method of treating diabetes mellitus. The method includes administering to a subject in need thereof an effective amount of any of the compound of formula (I) described above. “Treating” refers to administering one or more compounds described above to a subject, who has diabetes mellitus, a symptom of such a disease, or a predisposition toward such a disease, with the purpose to confer a therapeutic effect, e.g., to cure, relieve, alter, affect, ameliorate, or prevent diabetes mellitus, the symptom of it, or the predisposition toward it. “An effective amount” refers to the amount of one or more active compounds described above that is required to confer a therapeutic effect on a treated subject.

In still another aspect, this invention features a compound of formula (II): 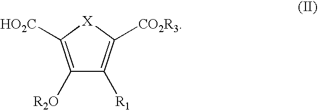
In this formula, X is O or S; R₁ is H, C₁-C₁₀ alkyl, C₃-C₂₀ cycloalkyl, C₃-C₂₀ heterocycloalkyl, aryl, heteroaryl, or ORₐ; R₂ is a hydroxyl protecting group; and R₃ is a carboxyl protecting group. Referring to formula (II), a subset of the just-described compounds are those in which R₁ is 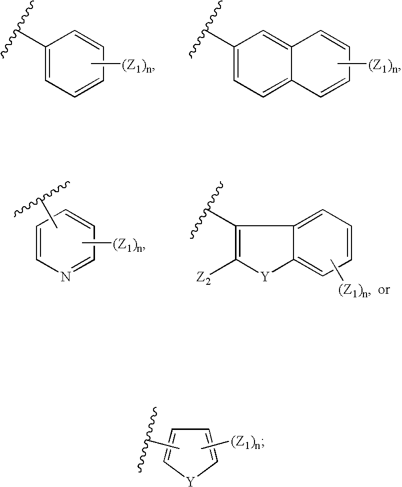
in which Y is O, S, N(R); n is 0-3; and each of Z₁ and Z₂, independently, is C₁-C₄ alkyl, C₃-C₇ cycloalkyl, C₃-C₇ heterocycloalkyl, aryl, heteroaryl, halogen, or OR′; each of R and R′, independently, being C₁-C₄ alkyl, C₃-C₇ cycloalkyl, C₃-C₇ heterocycloalkyl, aryl, or heteroaryl. In these compounds, R₂ and R₃, independently, can be unsubstituted C₁-C₁₀ alkyl (e.g., methyl), substituted C₁-C₁₀ alkyl (e.g., methoxymethyl or benzyl), or aryl. The compounds of formula (II) can be used as intermediates to prepare 4-hydroxyfuroic acid derivatives of formula (I).

In a further aspect, this invention features a method of preparing a compound of formula (II): 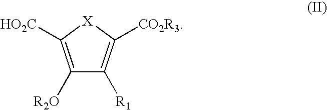
The method includes reacting a compound of formula (III): 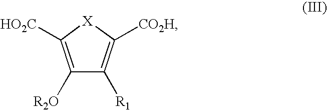
with R₄CO₂R₃ in a basic condition, and then with a tertiary amine; in which X is O or S; R₁ is H, C₁-C₁₀ alkyl, C₃-C₂₀ cycloalkyl, C₃-C₂₀ heterocycloalkyl, aryl, heteroaryl, or ORₐ; R₂ is a hydroxyl protecting group; R₃ is a carboxyl protecting group; and R₄ is halogen.

The 4-hydroxyfuroic acid derivatives described above include the compounds themselves, as well as their salts and their prodrugs, if applicable. A salt, for example, can be formed between an anion and a positively charged group (e.g., amino) on a 4-hydroxyfuroic acid derivative. Examples of suitable anions include chloride, bromide, iodide, sulfate, nitrate, phosphate, citrate, methanesulfonate, trifluoroacetate, and acetate. Likewise, a salt can also be formed between a cation and a negatively charged group (e.g., carboxylate) on a 4-hydroxyfuroic acid derivative. Examples of suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active 4-hydroxyfuroic acid derivatives.

Also within the scope of this invention is a composition containing one or more of the 4-hydroxyfuroic acid derivatives described above for use in treating diabetes mellitus, and the use of such a composition for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

Shown below are the structures of compounds 1-173, exemplary compounds of this invention:

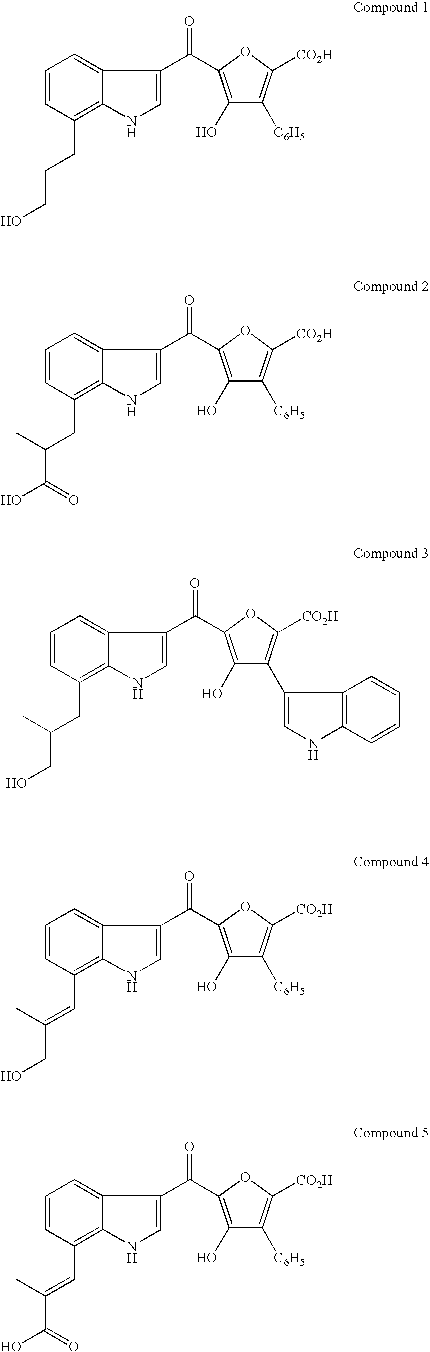

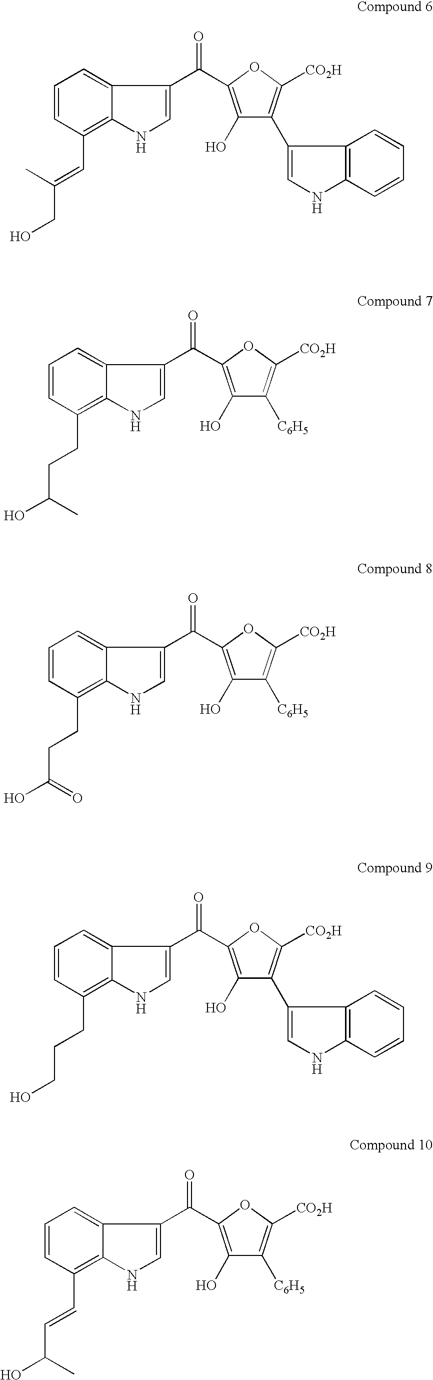

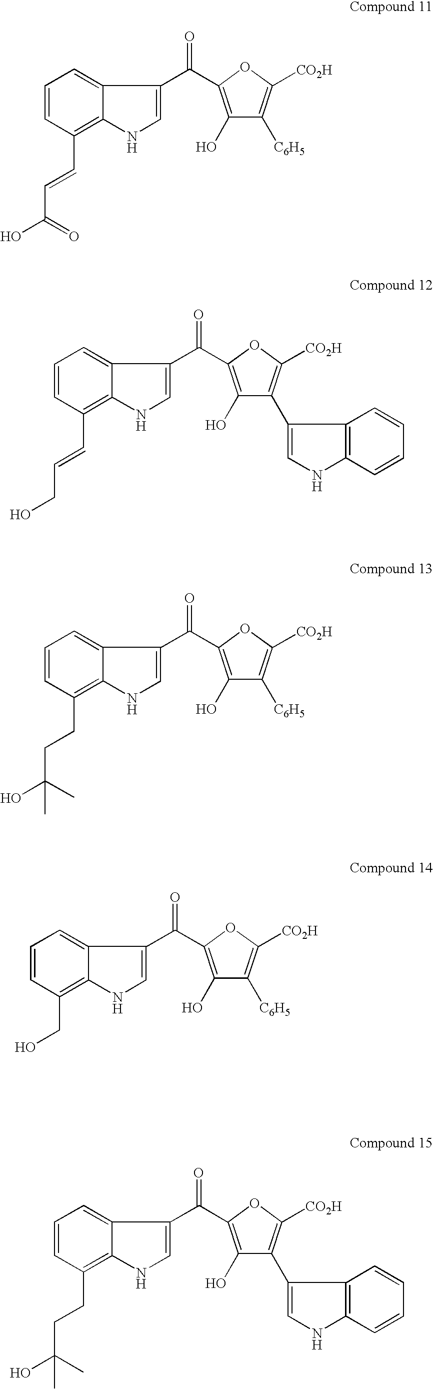

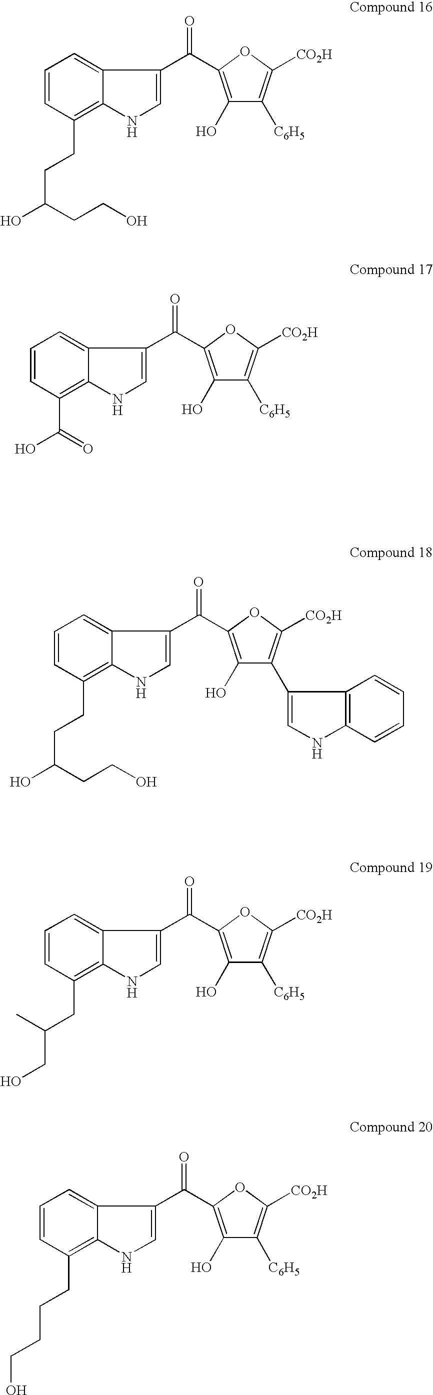

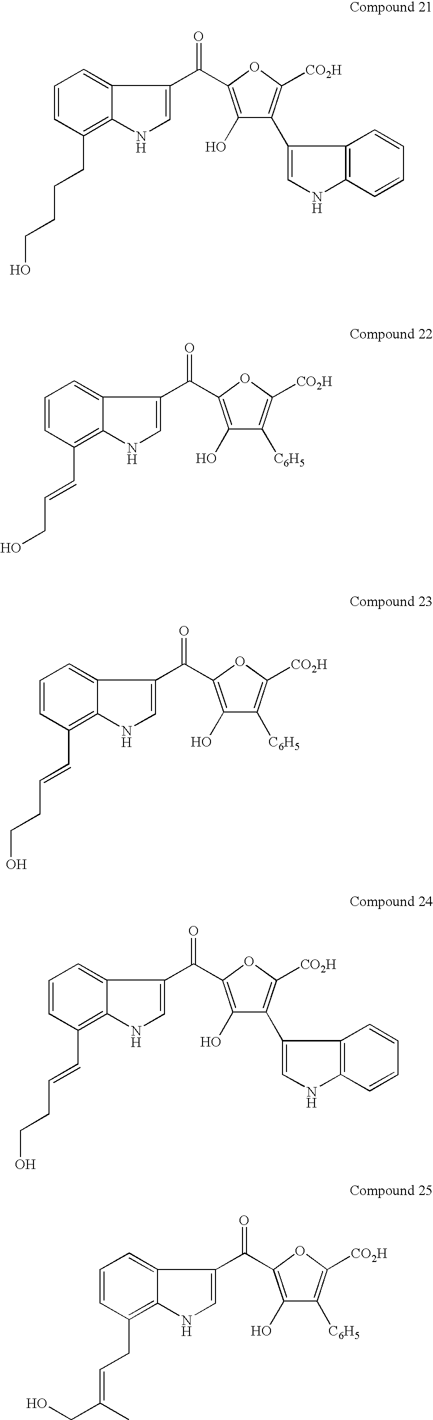

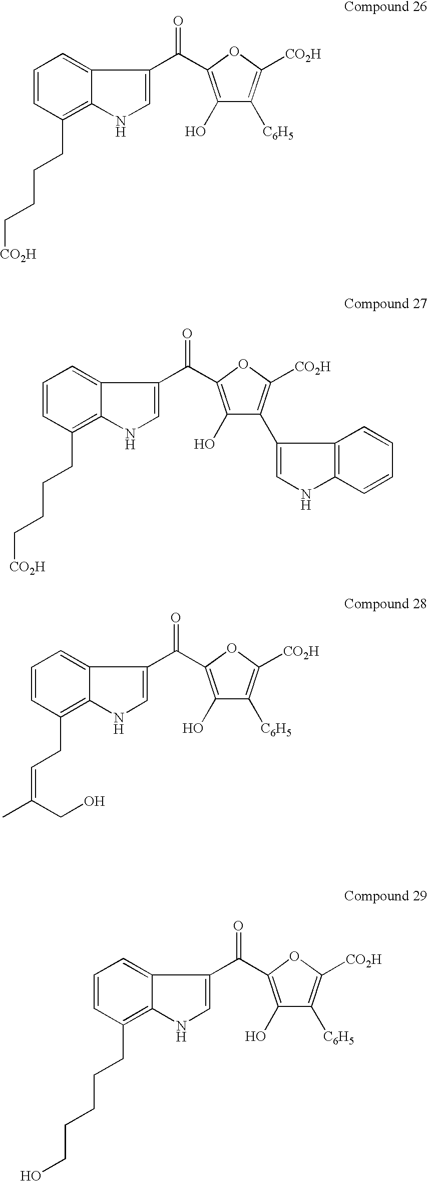

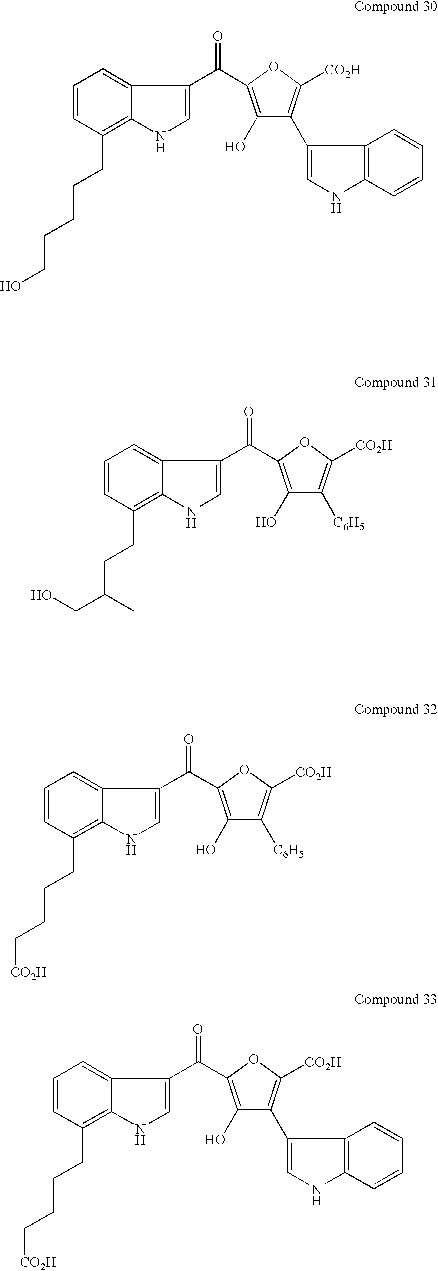

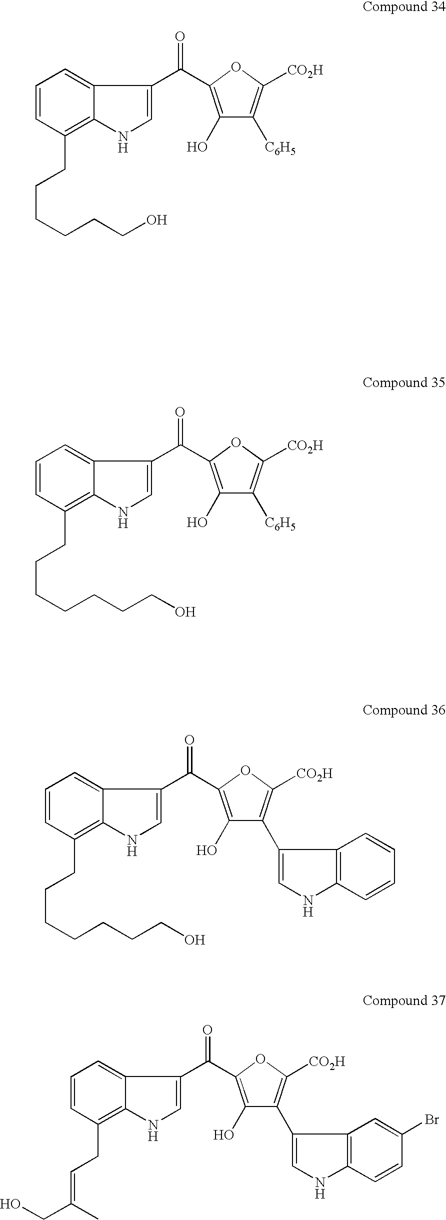

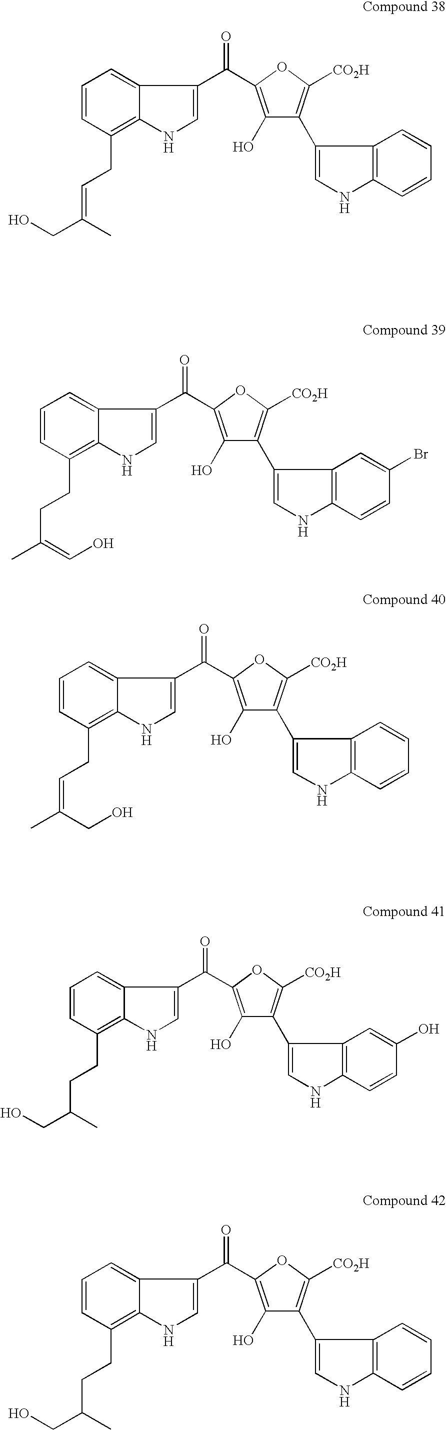

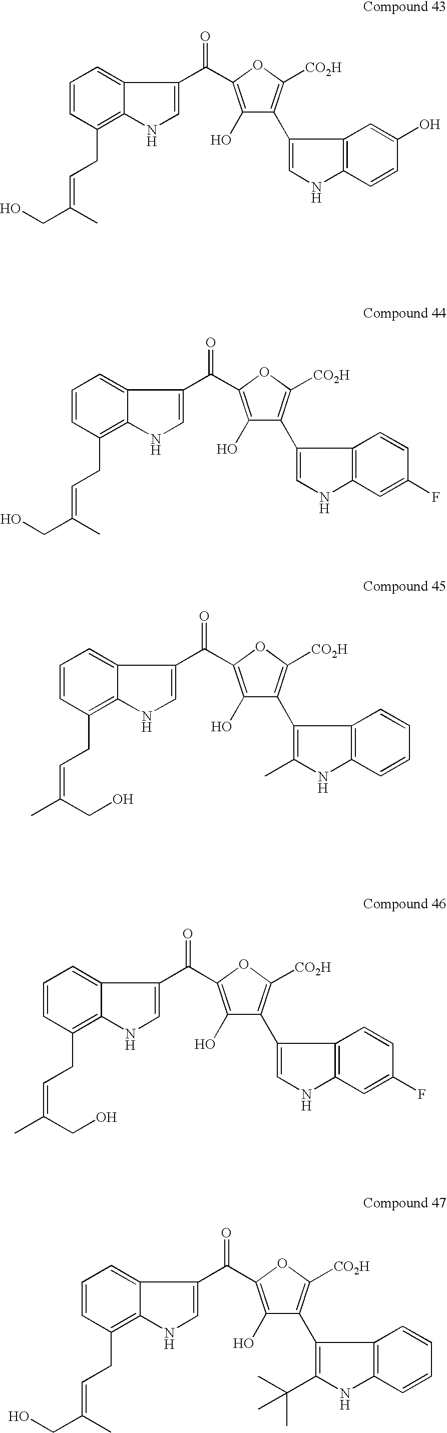

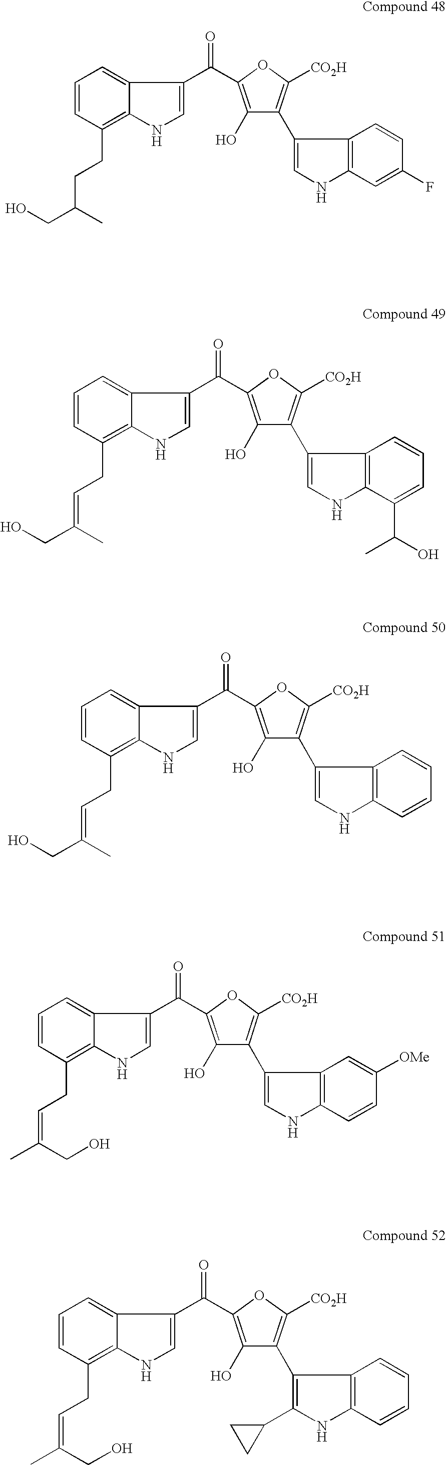

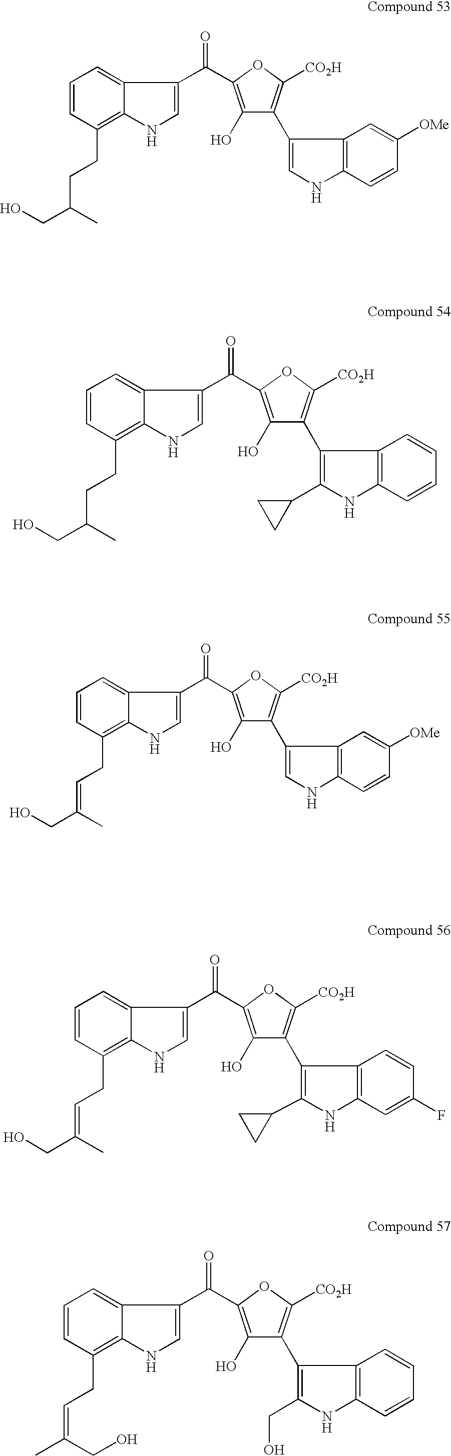

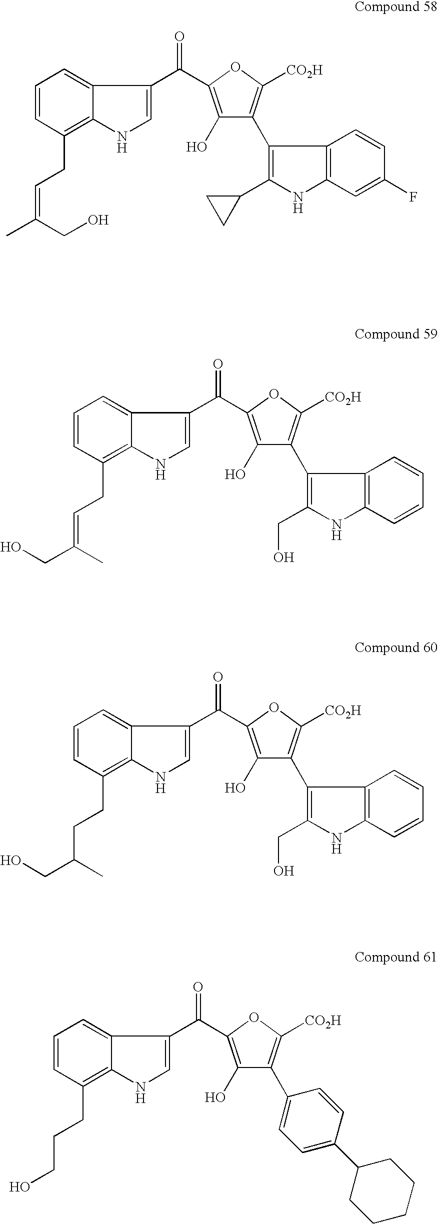

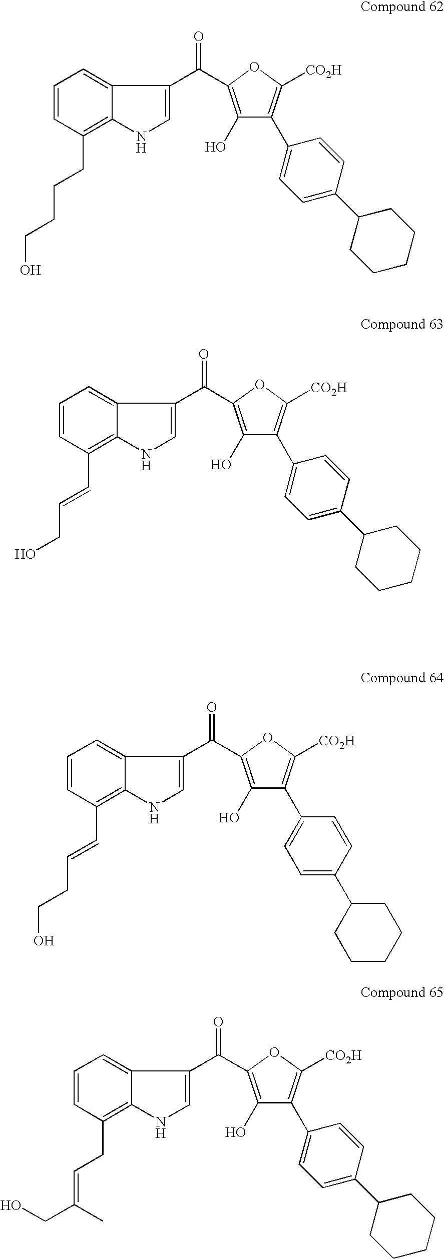

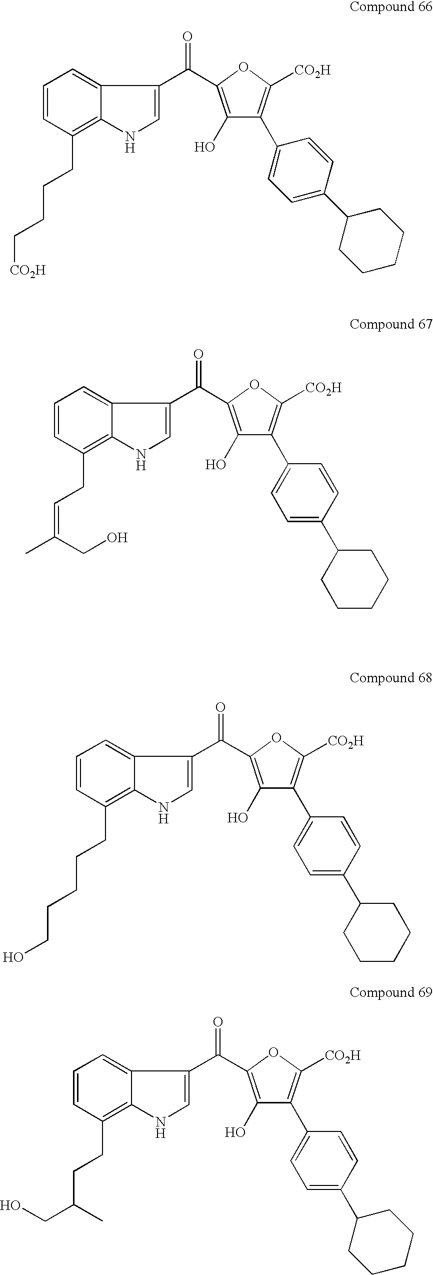

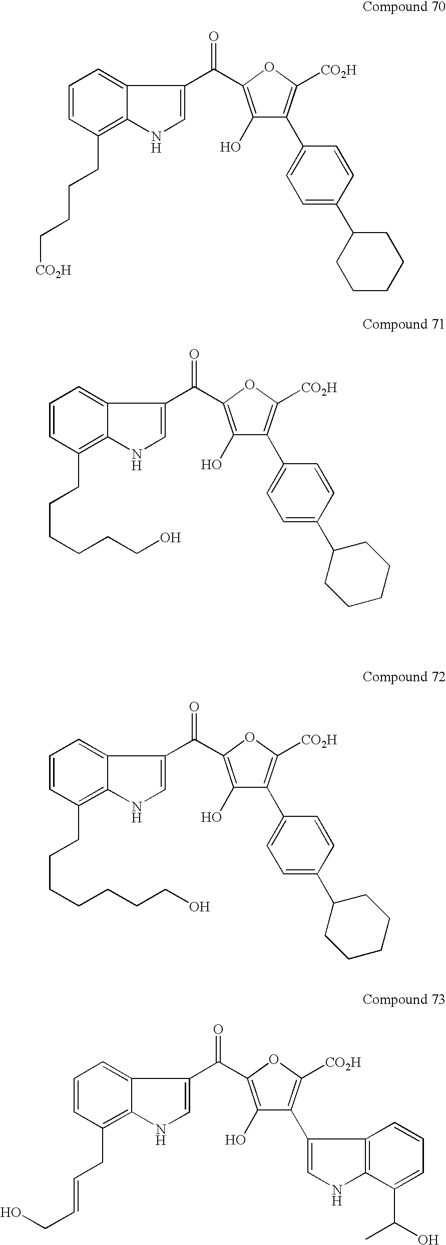

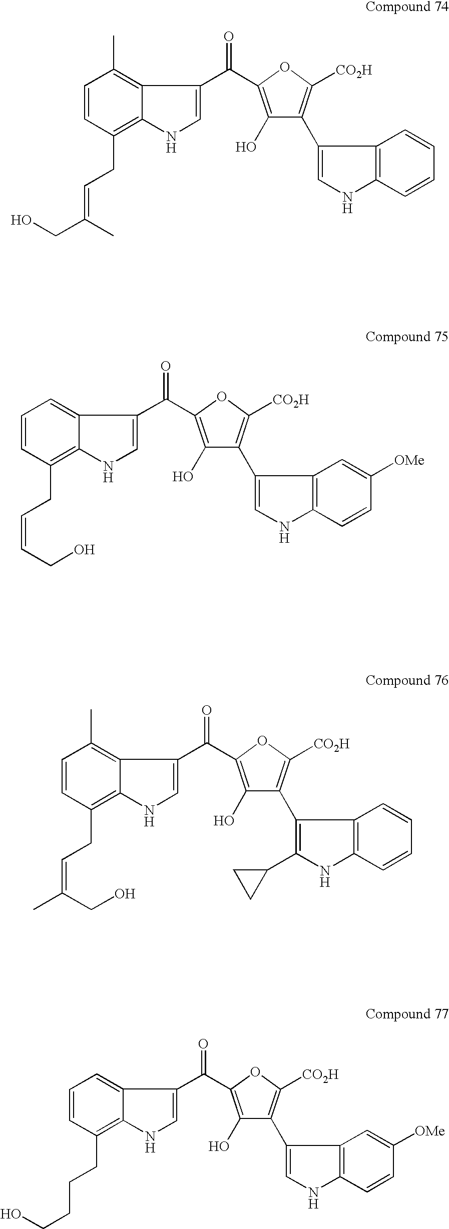

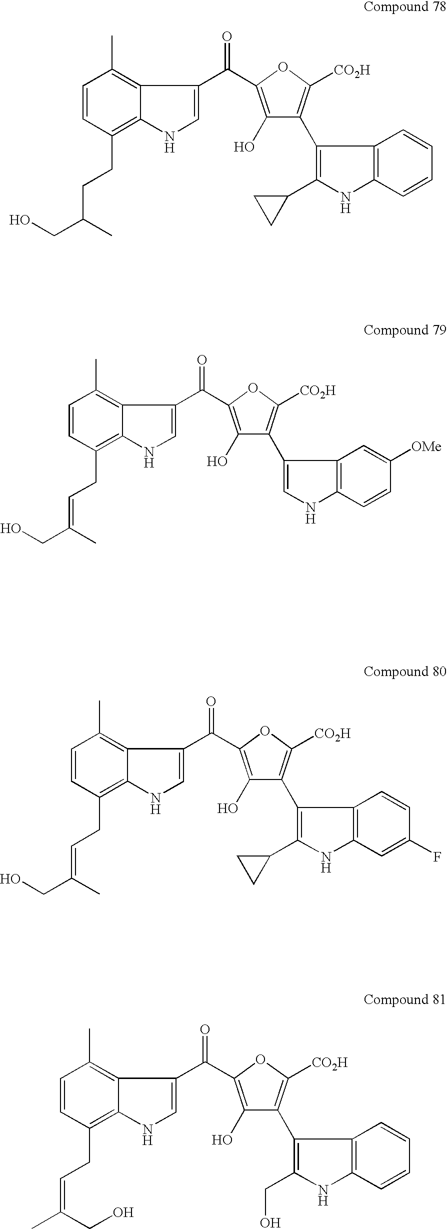

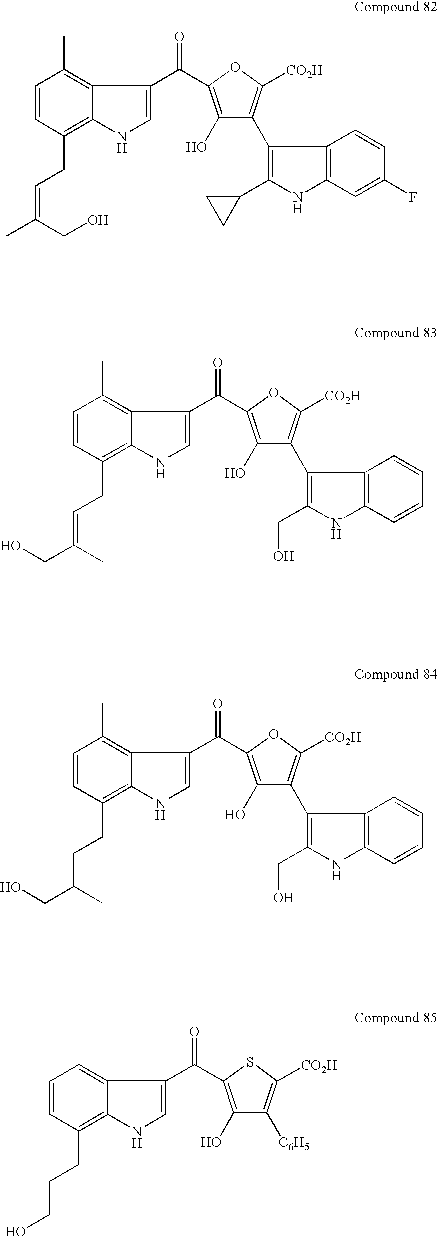

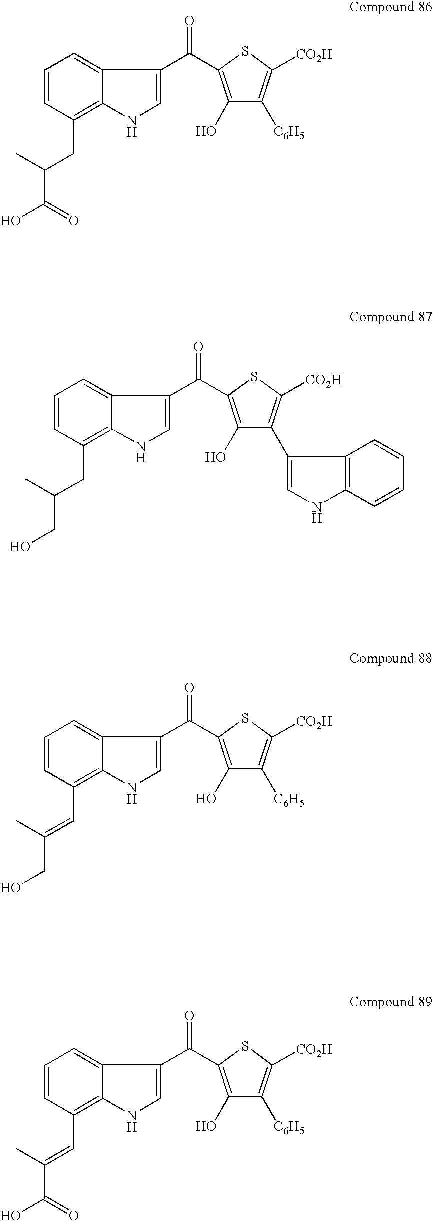

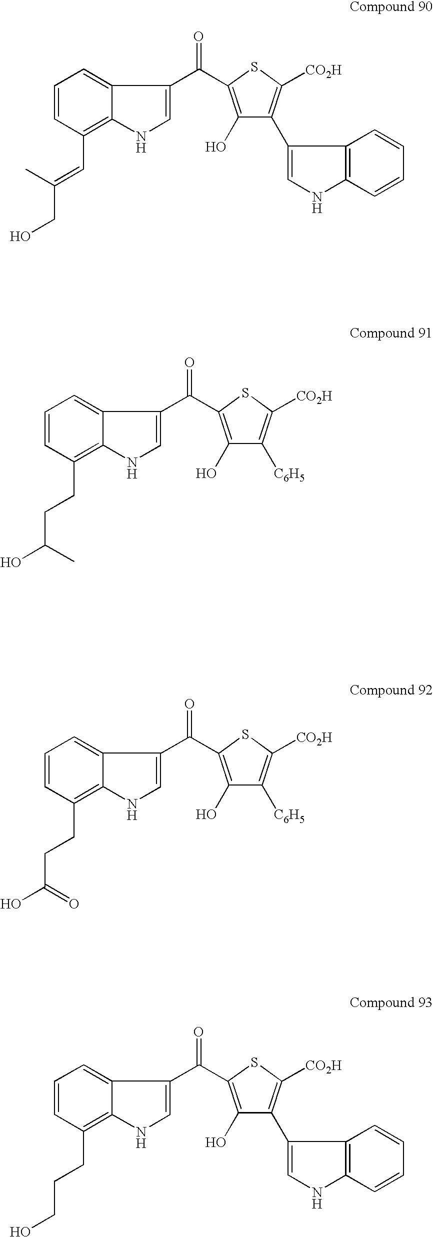

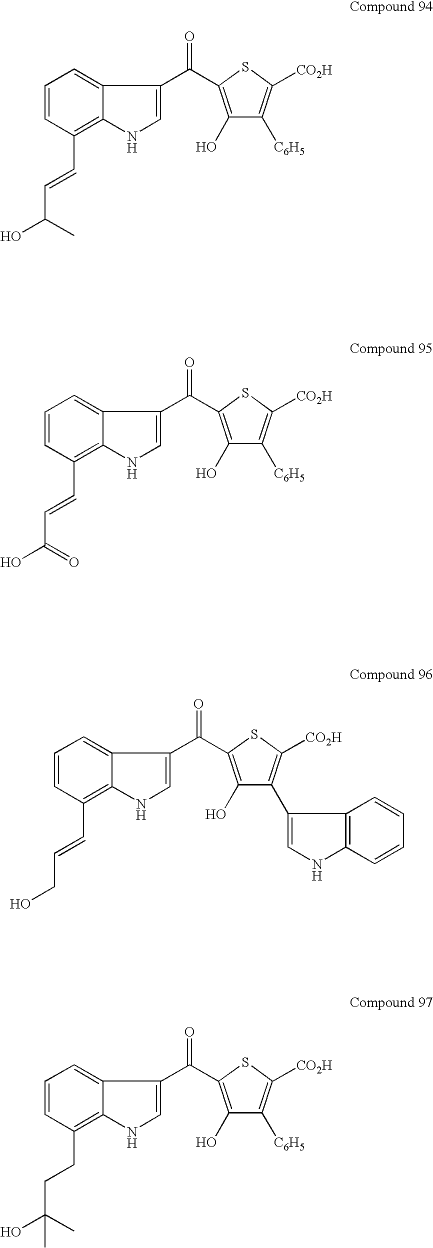

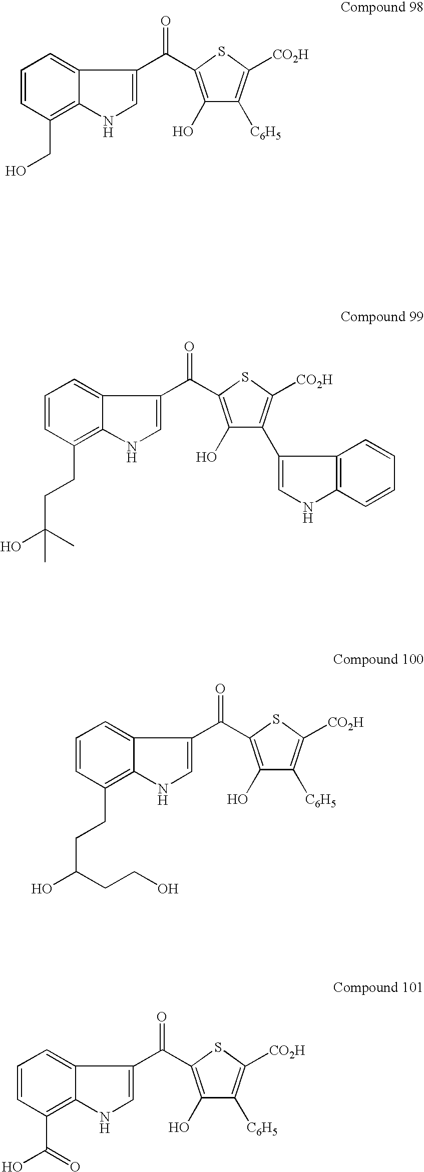

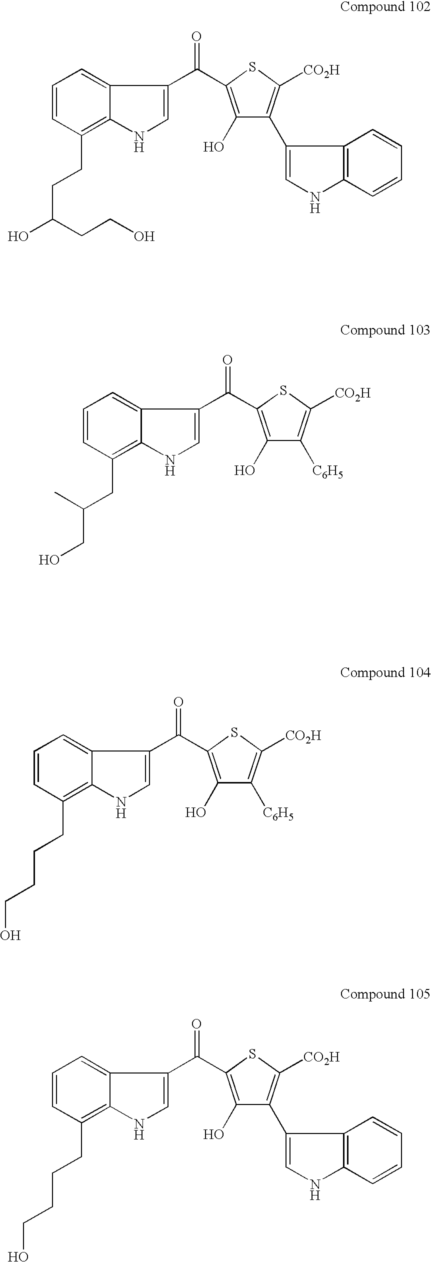

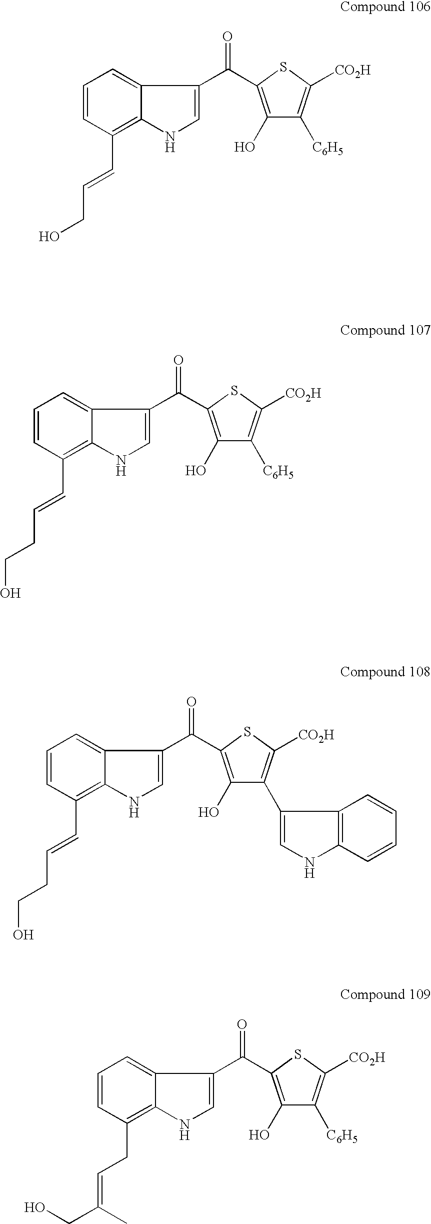

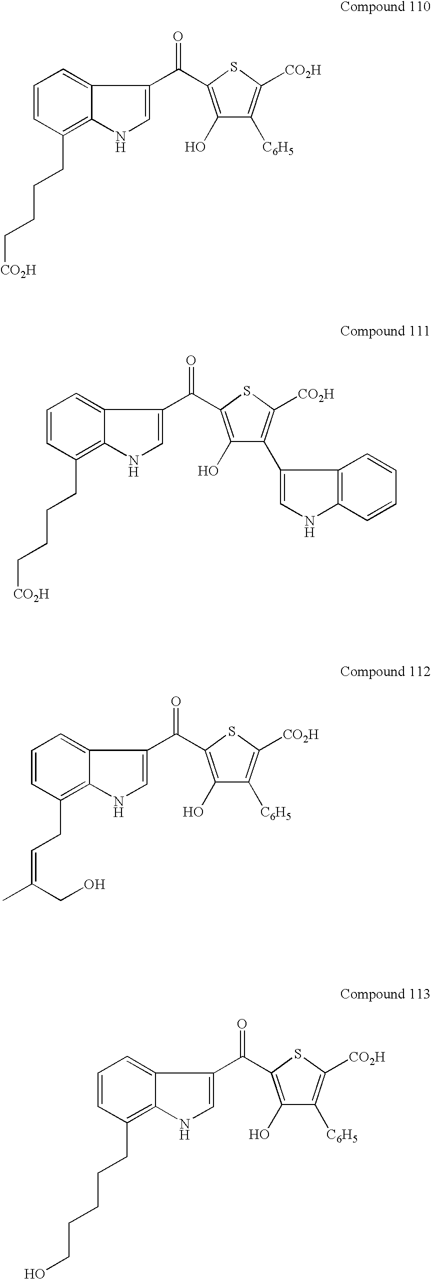

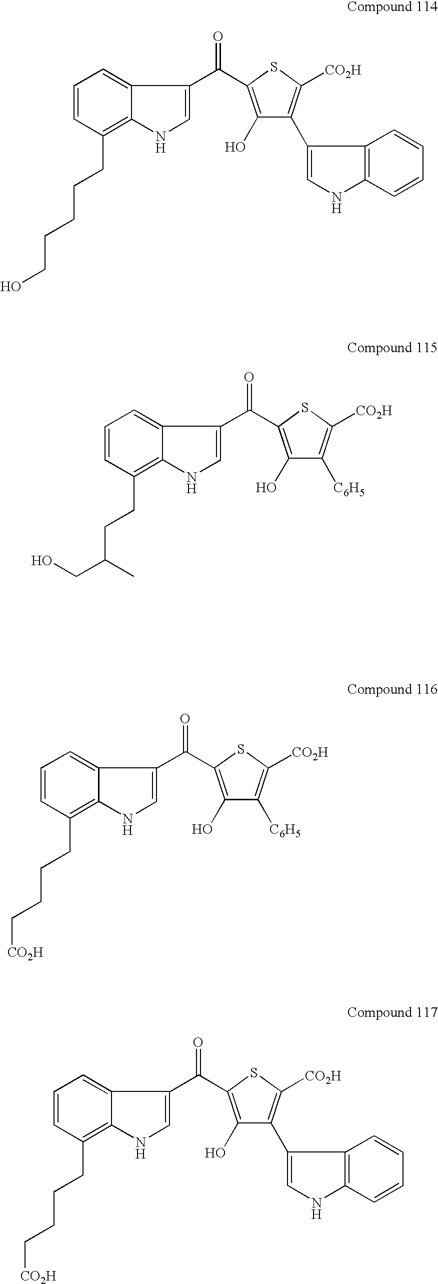

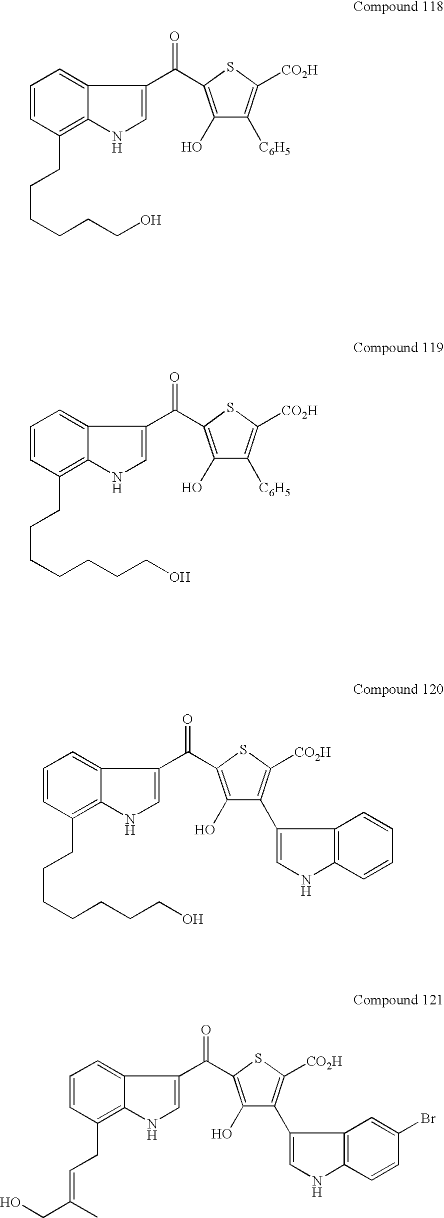

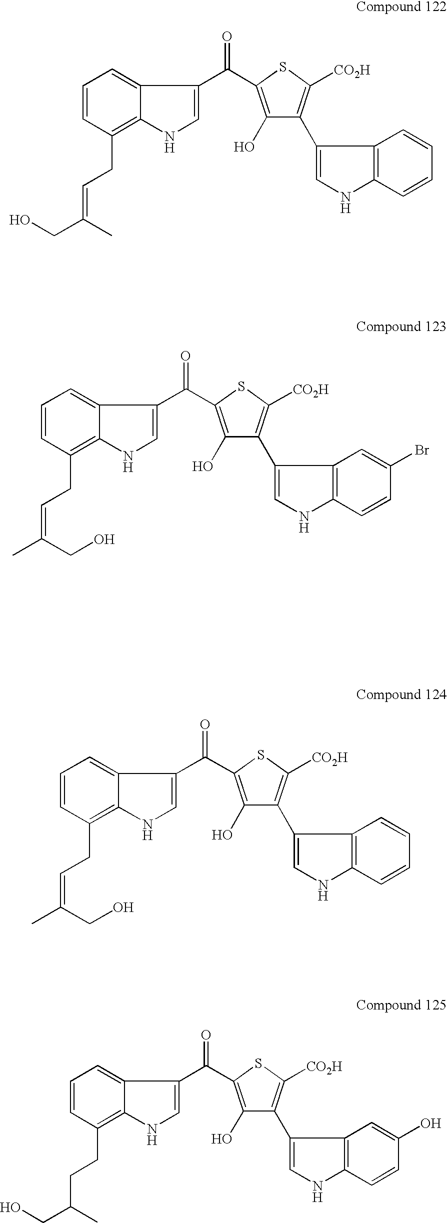

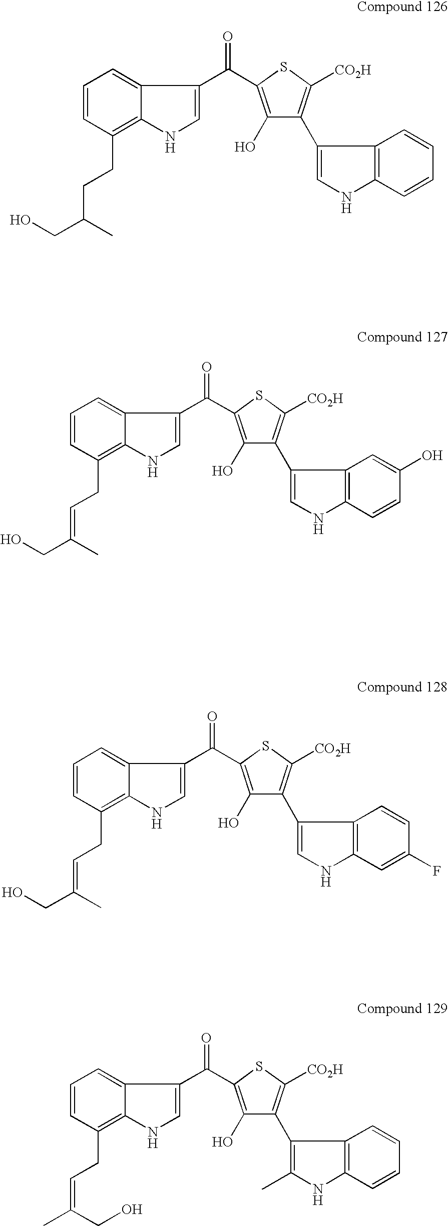

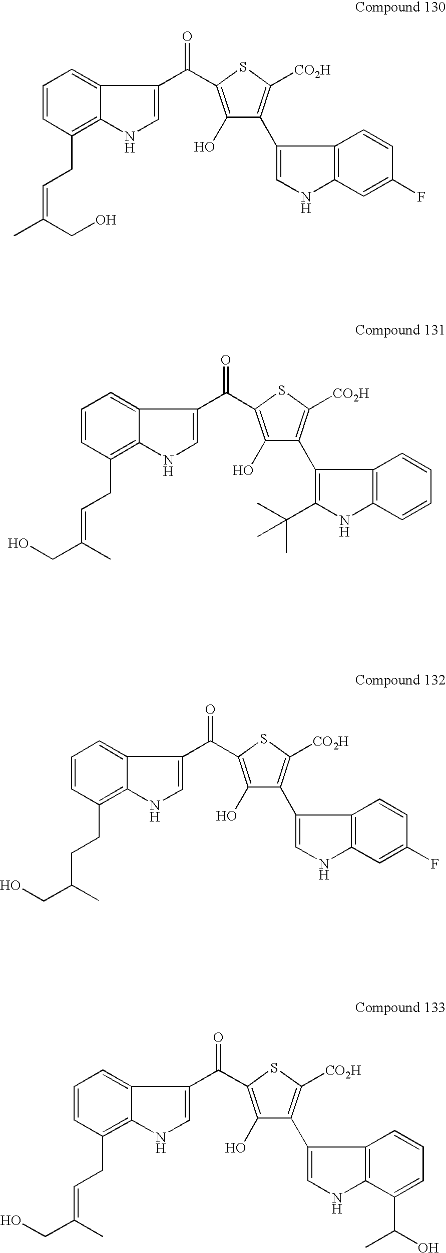

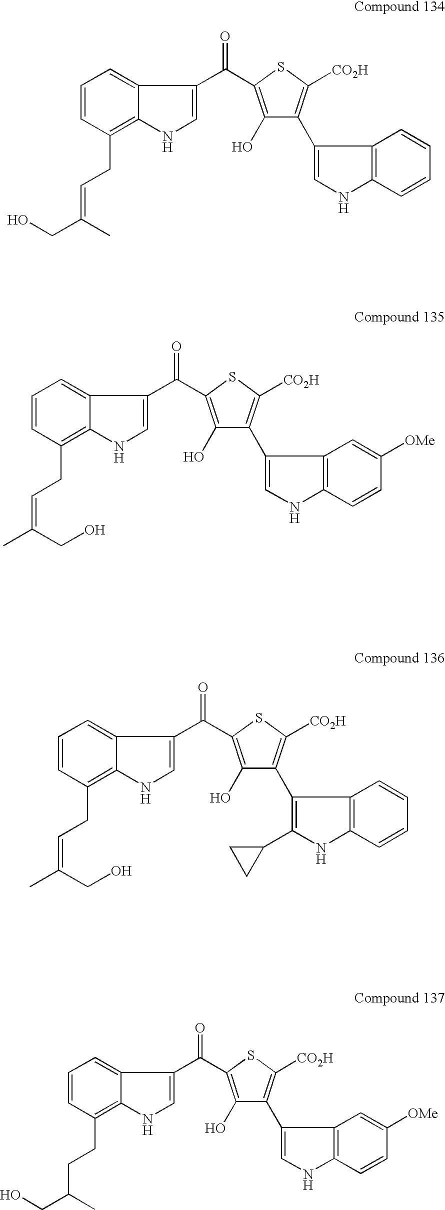

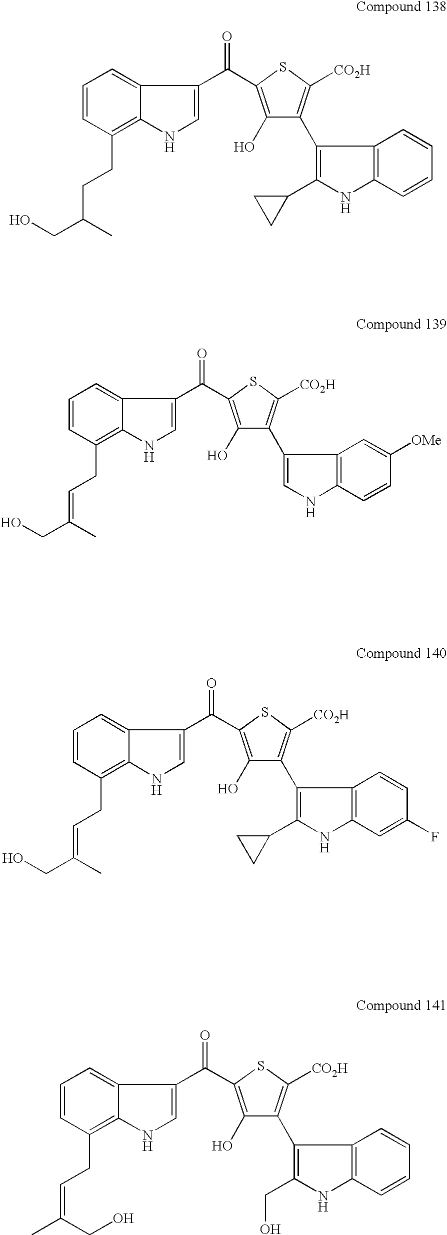

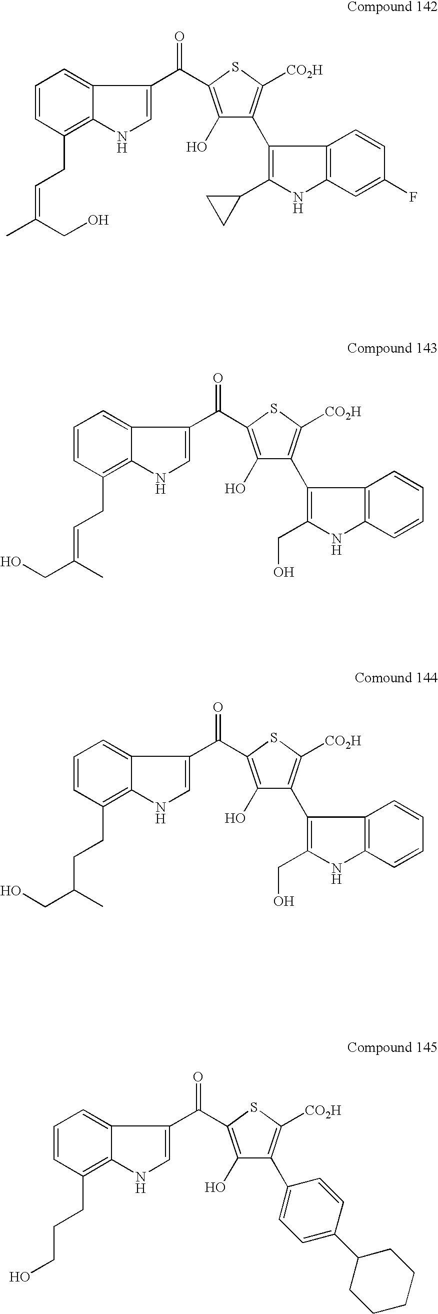

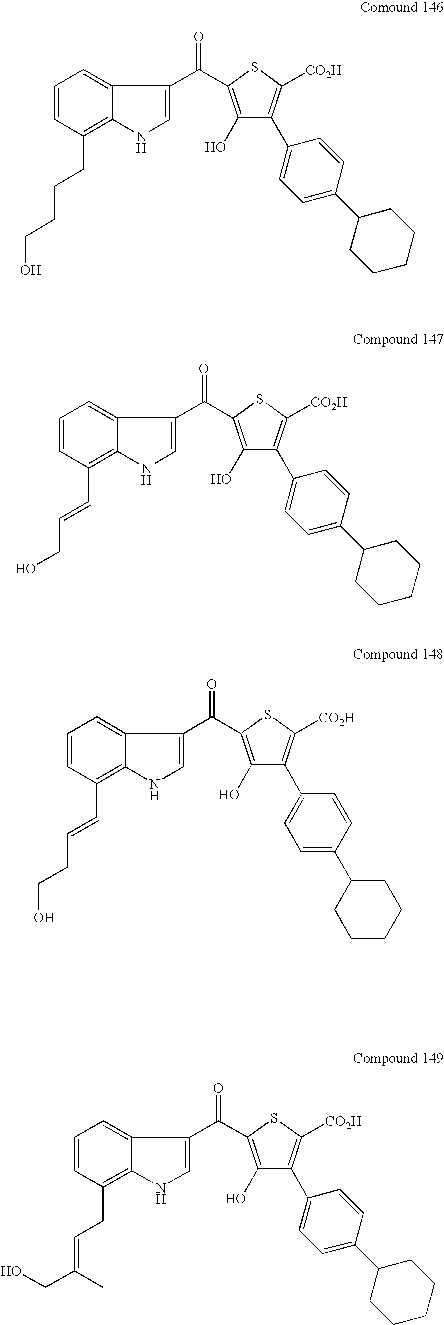

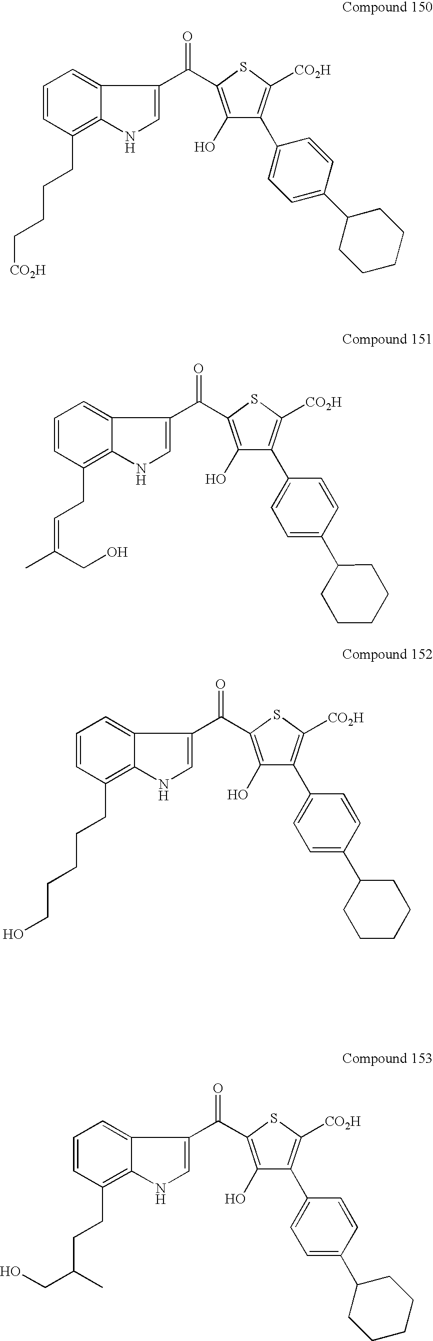

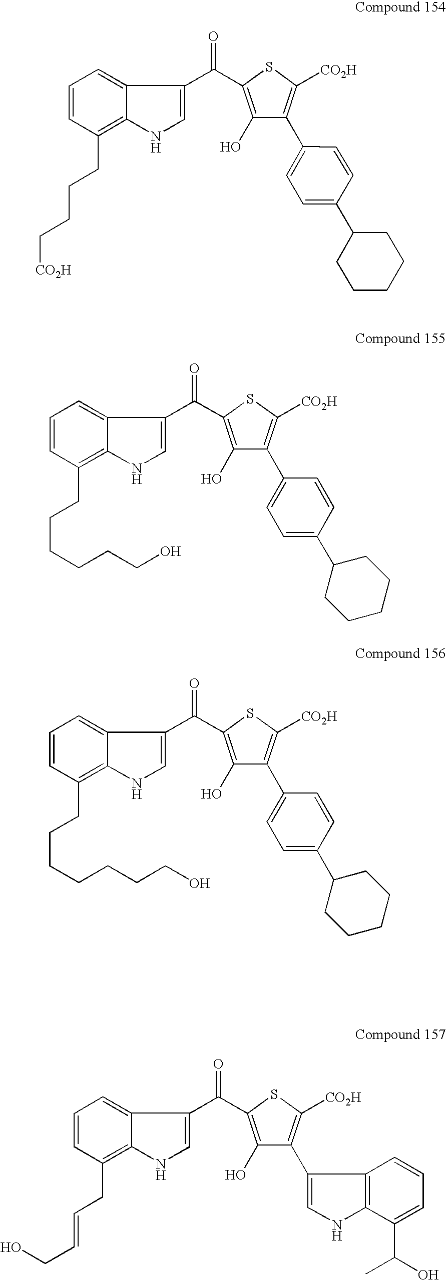

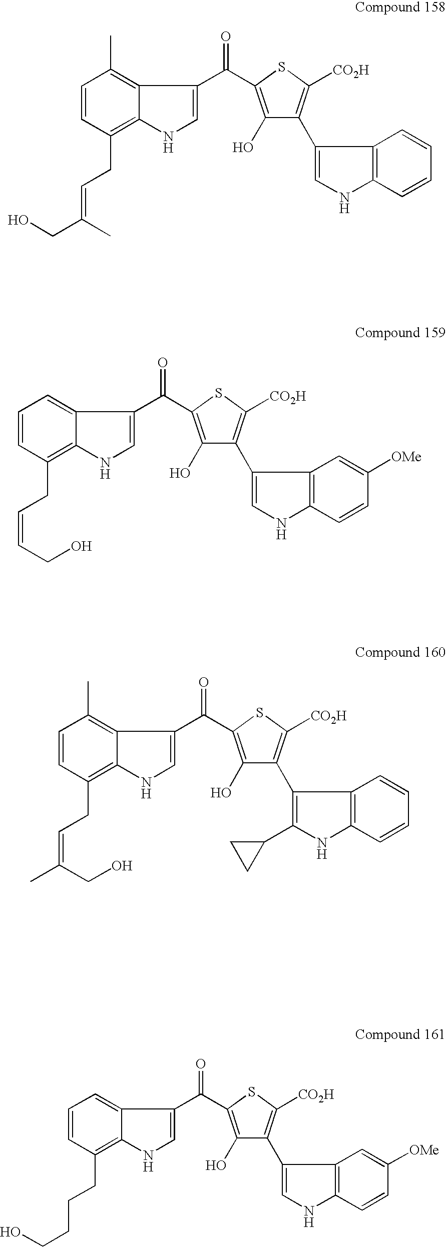

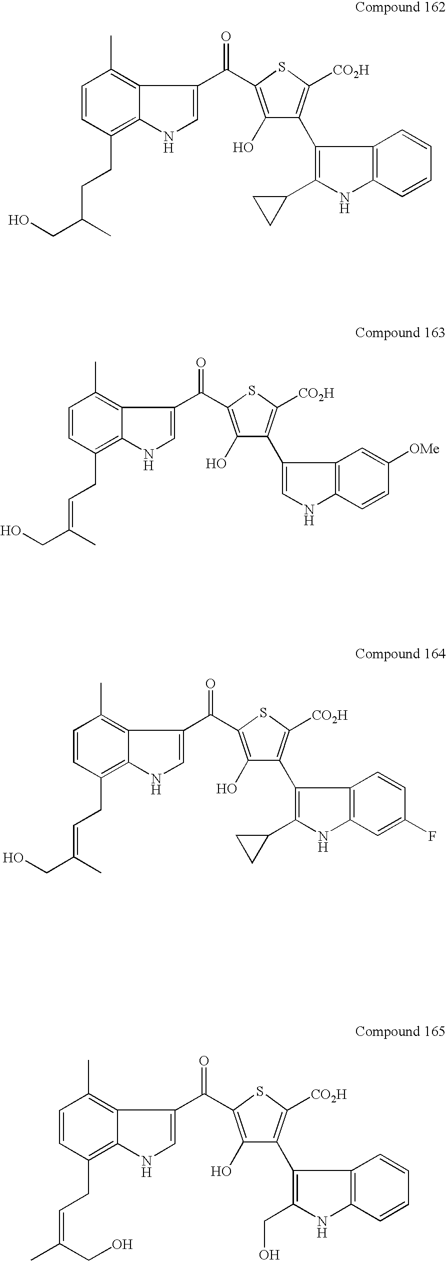

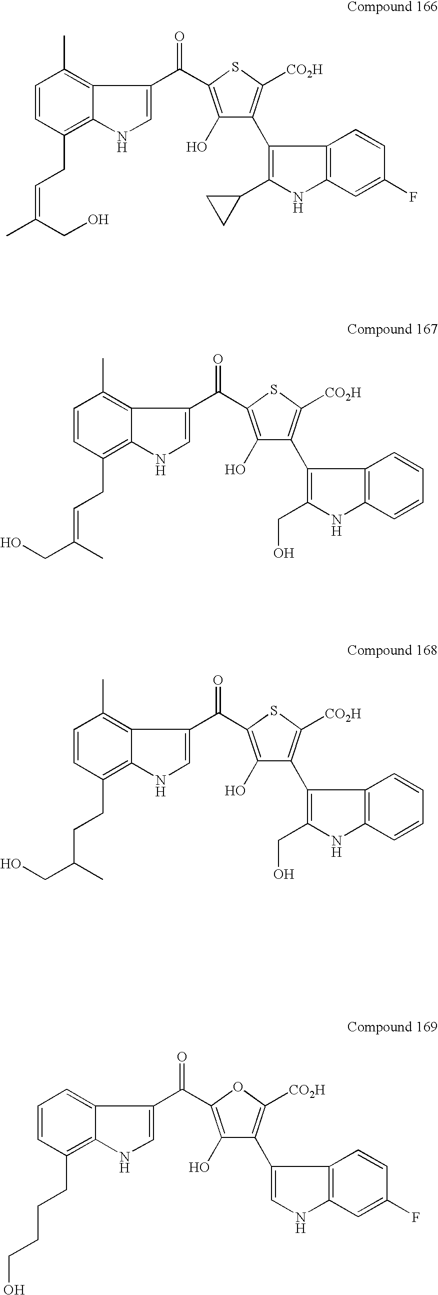

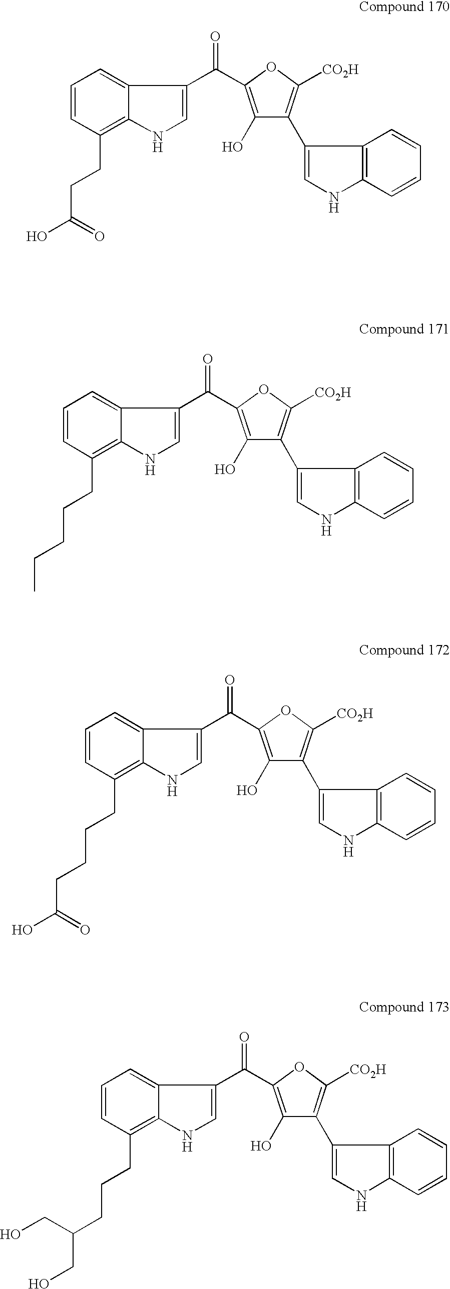

The 4-hydroxyfuroic acid derivatives described above can be prepared by methods well known to a skilled person in the art, as well as by the methods described herein. For example, Scheme 1 shown below depicts a typical route for synthesizing exemplary 4-hydroxyfuroic acid derivatives. Details of preparation of these compounds are provided in Examples 1-173.

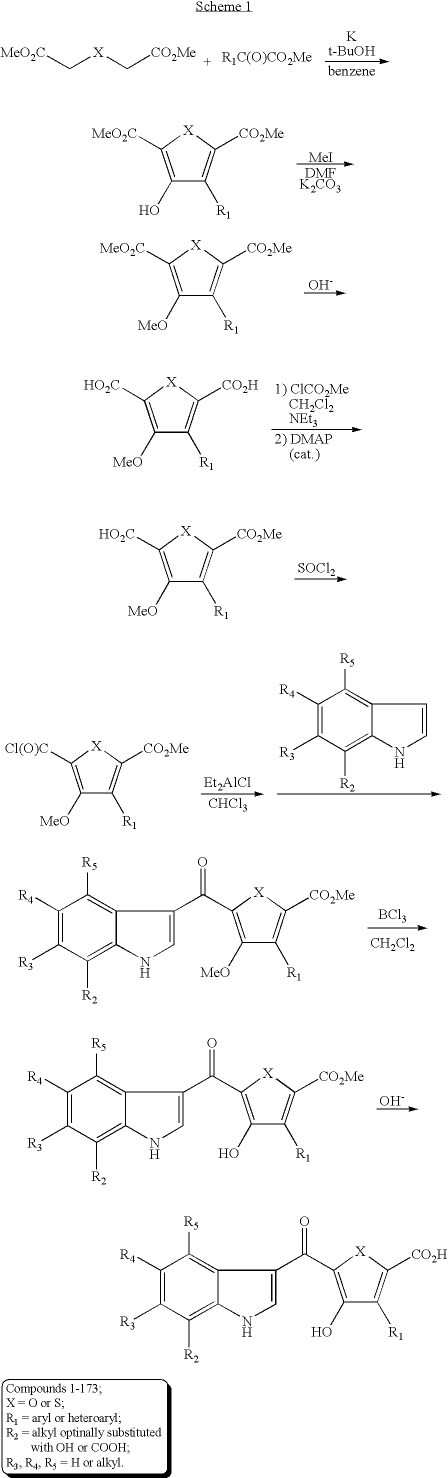

As shown in Scheme 1, a diester can react with a methyl formate to form a 4-hydroxyfuryl diester or a 4-hydroxythieno diester in the presence of a base. The hydroxy group on the diester can be protected by a hydroxyl-protecting group, such as a methyl group. The hydroxyl-protected diester can then undergo a hydrolysis reaction to form a diacid, followed by a regioselective methylation reaction to form a half ester, in which one of the carboxyl groups of the diacid is protected. The un-protected carboxyl group of the half ester can subsequently be converted to an acyl chloride group and then reacted with an indole compound. The compound thus obtained can then undergo deprotecting reactions to remove the hydroxyl-protecting group and the carboxyl-protecting group to afford a 4-hydroxyfuroic acid derivative of this invention. The just-mentioned indole compound can be prepared by known methods. For example, it can be prepared by reacting 7-formyl indole or substituted 7-formyl indole with a suitable ylid via a Wittig reaction or Hormer-Emmons reaction. If desired, the indole compound can be further modified by a transition metal-promoted carbon-carbon bond formation reaction. For example, an indole compound containing an allylacetate can be coupled with a malonate compound to prepare a diacid- or diol-containing indole compound. A substituted 7-formyl indole can be prepared by methods known in the art. For example, methoxy substituted 7-formyl indole compounds were obtained by Vilsmeier formylation at C-7 of the 4,6-dimethoxyindole compounds, which in turn were derived by a Bischler reaction. See, e.g., Black et al., J. Chem, Soc. Chem. Commun., 1985, 1172 and references cited therein.

Scheme 2 below depicts a typical regioselective method of prepare a half ester, an intermediate for preparing a 4-hydroxyfuroic acid derivative of this invention. Specifically, a diacid can be first treated with a chloroformate in a basic condition, and then with a catalytic amount of a tertiary amine (e.g., DMAP or pyridine) to form a mixture of two isomers. Unexpectedly, the desired half ester isomer is the major component of the mixture obtained from this regioselective method. By contrast, a conventional method, such as hydrolysis of a corresponding diester, can only provide a mixture in which the undesired half ester is the major component.

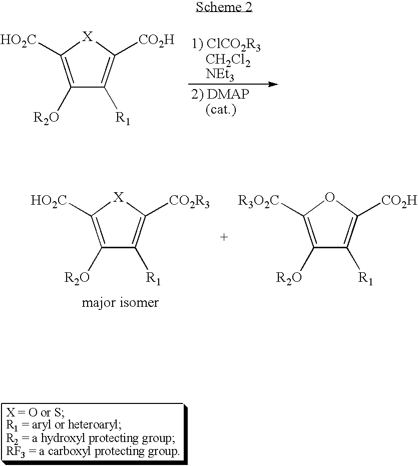

Other 4-hydroxyfuroic acid derivatives can be prepared using other suitable starting materials following the synthetic routes disclosed herein and other synthetic methods known in the art.

The methods described above may also additionally include steps, either before or after the steps described specifically herein, to add or remove suitable protecting groups in order to ultimately allow synthesis of the 4-hydroxyfuroic acid derivatives. For example, to synthesize a 4-hydroxyfuroic acid derivative in which R₁ is an indole moiety, the amino group in the indole moiety can be protected during the synthesis and deprotected in the last step to form the desired compound. The indolyl amino group can also be protected sequentially with different protecting groups. For example, to prepare a 4-hydroxyfuryl diester intermediate, a starting material, (1H-indol-3-yl)-oxo-acetic acid methyl ester, can be protected by benzyl, 4-nitrobenzyl, or 4-methoxybenzyl. After the hydroxyl group in the resultant diester is protected (e.g., by methylation), the amino-protecting group can be replaced with an electron withdrawing protecting group (e.g., tosyl, substituted tosyl, or mesyl) by routine methods known in the art. The resultant intermediate can then undergo ester hydrolysis and selective half ester formation (see Scheme 2) to provide a desired half ester. Such a half ester facilitates the subsequent acylation reaction by minimizing non-selective acylation. In addition, various synthetic steps may be performed in an alternate sequence or order to give the desired compounds. Synthetic chemistry transformations and protecting group methodologies (protection and deprotection) useful in synthesizing applicable 4-hydroxyfuroic acid derivatives are known in the art and include, for example, those described in R. Larock, Comprehensive Organic Transformations, VCH Publishers (1989); T. W. Greene and P. G. M. Wuts, Protective Groups in Organic Synthesis, 2nd Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The 4-hydroxyfuroic acid derivatives mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Also within the scope of this invention is a pharmaceutical composition contains an effective amount of at least one 4-hydroxyfuroic acid derivatives described above and a pharmaceutical acceptable carrier. Further, this invention covers a method of administering an effective amount of one or more of the 4-hydroxyfuroic acid derivatives to a patient with diabetes mellitus. Effective doses will vary, as recognized by those skilled in the art, depending on the types of diseases treated, route of administration, excipient usage, and the possibility of co-usage with other therapeutic treatment.

To practice the method of the present invention, a composition having one or more 4-hydroxyfuroic acid derivatives can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more active 4-hydroxyfuroic acid derivatives can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active 4-hydroxyfuroic acid derivatives. Examples of other carriers include colloidal silicon oxide, magnesium stearate, cellulose, sodium lauryl sulfate, and D&C Yellow # 10.

The 4-hydroxyfuroic acid derivatives described above can be preliminarily screened for their efficacy in treating diabetes mellitus by in vitro assays (See Examples 170 and 171 below) and then confirmed by animal experiments and clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of Compound 1

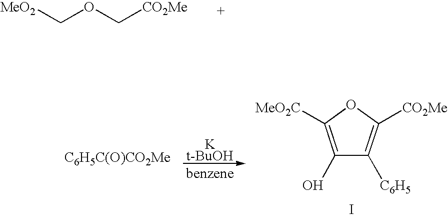

A suspension of potassium t-butoxide was prepared by reacting potassium (1.6 g, 0.041 mole) and t-butanol (3 g) in benzene (40 mL). The suspension was then kept under reflux. A solution of dimethyl diglycolic acid ester (2.42 g, 0.015 mol) and methyl benzoyl formate (3.00 g, 0.02 mol) in benzene (10 mL) was added dropwise to the above refluxing suspension. After being refluxed under nitrogen for 2.5 hours, the mixture was cooled, diluted with ice water, and acidified with 10% sulfuric acid. The resultant mixture was extracted with ethyl acetate. The extracts were combined, washed with water, dried, and concentrated to give a crude product. The crude product was purified by silica gel column chromatography using 1/4 ethyl acetate-hexane as an eluent to afford Intermediate I (2.2 g, 53.3%). ¹H-NMR (CDCl₃): 7.60 (m, 2H), 7.47 (m, 3H), 4.02 (s, 3H), 3.88 (s, 3H). LC/MS (M⁺+1): 277.2.

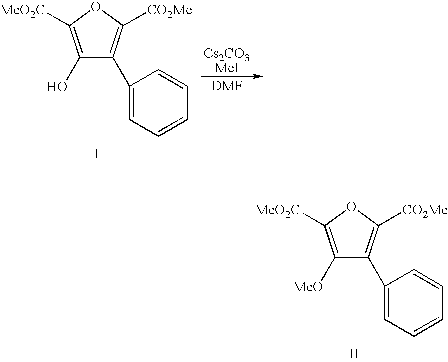

Cs₂CO₃ (1.36 g, 4.2 mmol) and MeI (0.4 g, 2.8 mmol) were added sequentially to a solution of Intermediate I (0.56 g, 2.0 mmol) in DMF (10 mL). After being stirred at 40° C. for 21 hours, the reaction mixture was filtered from a Sintered glass funnel. The filtrate was concentrated under vacuum below 50° C. The residue thus obtained was diluted with dichloromethane, and washed with brine and water. The organic layer was collected, dried with MgSO₄, and concentrated to give a crude product. The crude product was purified by silica gel column chromatography using 1/4 ethyl acetate-hexane as an eluent to afford Intermediate II (0.58 g, 98.6%). ¹H-NMR (CDCl₃): 7.46 (m, 5H), 3.96 (s, 3H), 3.83 (s, 3H). LC/MS (M⁺+1): 291.5.

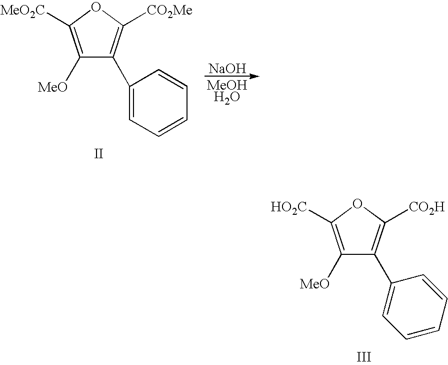

Intermediate II was refluxed in a solution containing 5% sodium hydroxide in methanol to afford Intermediate III in a 92% yield. ¹H-NMR (⁶d-acetone): 7.54 (m, 2H), 7.45 (m, 3H), 3.89 (s, 3H).

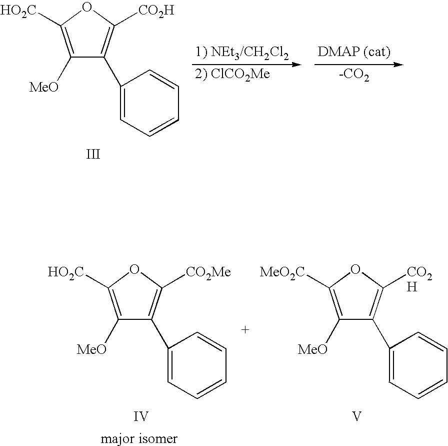

To a solution of Intermediate III (0.50 g, 1.9 mmol) and triethylamine (0.29 g, 2.85 mmol, 150 mole % vs Intermediate III) in dichloromethane (10 mL) at 0° C. was added a solution of methyl chloroformate (0.18 g, 1.9 mmol, 100 mole % vs Intermediate III) in dichloromethane (2 mL). After stirring at 0° C. for 60 minutes, DMAP (42 mg) was added. The resulting solution was stirred at 0° C. for 1 hour and at room temperature for additional 18 hours. The reaction mixture was diluted with dichloromethane (20 mL) and extracted with a Na₂CO₃ solution (20 mL×2). The extracts were combined, washed with ether, and then acidified with a 3 N HCl solution. The acidified mixture was extracted with ethyl acetate (50 mL×2). The extracts was combined, washed twice with water, dried with MgSO₄, and concentrated to give a mixture of intermediates IV and V (0.31 g, ˜59%) as a white powder. HPLC analysis showed that the ratio of intermediate IV: Intermediate V was about 4.8:1. The ratio was confirmed by ¹H NMR by comparing the signal ratio between the ester methyl groups appeared at 3.78 ppm and 3.92 ppm, respectively. Intermediates IV and V was isolated from the mixture by silica gel column chromatography using 1/4 to 1/2 ethyl acetate/hexane as an eluent. Intermediate IV: ¹H NMR (⁶d-acetone): 7.52 (m, 2H), 7.43 (m, 3H), 3.87 (s, 3H), 3.78 (s, 3H). Intermediate V: ¹H NMR (⁶d-acetone): 7.52 (m, 2H), 7.43 (m, 3H), 3.92 (s, 3H), 3.87 (s, 3H).

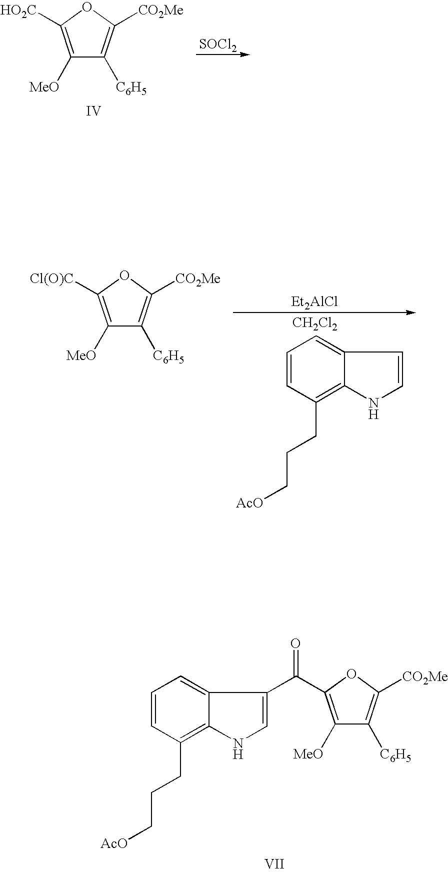

Thionyl chloride (3.5 mL) was added to Intermediate IV (0.33 g, 1.22 mmol). After being refluxed for 20 minutes, the reaction mixture was concentrated and dried under vacuum to afford Intermediate VI as a viscous oil.

To a dichloromethane solution (6 mL) of acetic acid 3-(1H-indol-7-yl)-propyl ester (0.27 g, 1.24 mmol) was added 3.0 mL (3.0 mmol) of Et₂AlCl (1M in hexane) at 0° C. The mixture was stirred at this temperature for 0.5 hours. A dichloromethane solution (5 mL) of Intermediate VI was then added to this mixture dropwise at 0° C. The resultant mixture was then stirred at 0° C. for 1 hour and at room temperature for 14 hours. The reaction mixture was diluted with 30 mL of dichloromethane and quenched with 25 mL of ice water and 10 mL of 10% H₂SO₄. The mixture was then filtered from Celite. The organic layer was washed sequentially with brine and water, dried, and concentrated to give a dark brown residue. The residue thus obtained was purified by silica gel column chromatography using 1/3 ethyl acetate-hexane as an eluent to afford Intermediate VII. ¹H-NMR (⁶d-acetone): 8.66 (d, J=3.2 Hz, 1H), 8.40 (d, J=7.8 Hz, 1H), 7.57 (m, 2H), 7.46 (m, 3H), 7.25 (m, 1H), 7.17 (d, J=7.1 Hz, 1H), 4.13 (m, 2H), 3.96 (s, 3H), 3.82 (s, 3H), 3.08 (t, J=7.8 Hz, 2H), 2.10 (m, 2H).

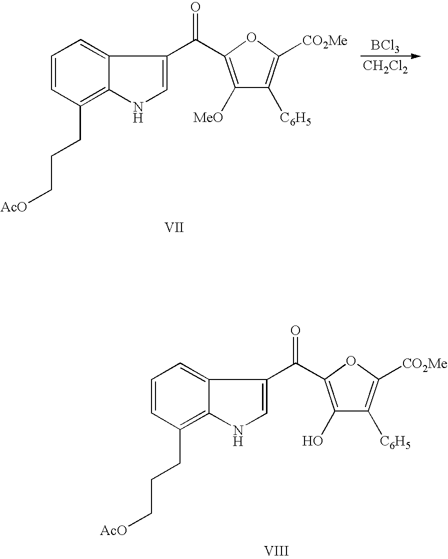

To a dichloromethane solution (5.0 mL) of Intermediate VII (0.25 g, 0.53 mmol) was added 3.5 mL (3.5 mmol) of BCl₃ (1M in hexane) at 0° C. The reaction mixture was stirred at 0° C. for 0.5 hours and then at room temperature for 3 hours. Dichloromethane (20 mL) was added and the mixture was stirred for another 0.5 hours. The mixture was then quenched with 25 mL of ice water and extracted with dichloromethane. The organic layer was collected, washed sequentially with brine and water, dried, and concentrated to give a dark brown residue. The residue thus obtained was purified by silica gel column chromatography using 1/3 ethyl acetate-hexane as an eluent to afford Intermediate VIII. ¹H-NMR (⁶d-acetone): 8.76 (d, J=3.2 Hz, 1H), 8.35 (d, J=7.8 Hz, 1H), 7.67 (m, 2H), 7.47 (m, 3H), 7.27 (m, 1H), 7.20 (d, J=7.2 Hz, 1H), 4.13 (d, J=6.5 Hz, 2H), 3.87 (s, 3H), 3.08 (t, J=7.7 Hz, 2H), 2.10 (m, 2H).

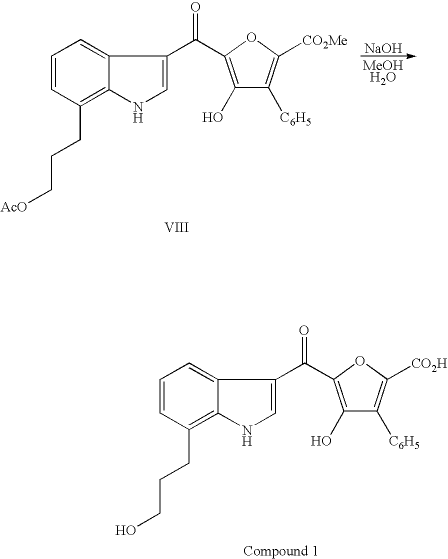

To a methanol solution (2.0 mL) of Intermeidate VIII (0.11 g) was added 1.0 mL of a 5% sodium hydroxide aqueous solution. The mixture was refluxed for 0.5 hours. It was then concentrated, acidified with 10% sulfuric acid, and extracted with ethyl acetate. The organic layer was collected, washed sequentially with brine and water, dried, and concentrated to give a dark brown residue. The residue was triturated with diethyl ether and filtered to afford Compound 1 as a yellow powder. ¹H-NMR (⁶d-acetone): 8.80 (d, J=3.2 Hz, 1H), 8.35 (d, J=7.9 Hz, 1H), 7.71 (m, 2H), 7.45 (m, 3H), 7.25 (m, 1H), 7.18 (d, J=7.1 Hz, 1H), 3.64 (t, J=6.2 Hz, 2H), 3.06 (t, J=7.5 Hz, 2H), 1.97 (m, 2H). LC/MS (M⁺+1): 406.0.

### EXAMPLE 2

### Preparation of Compound 9

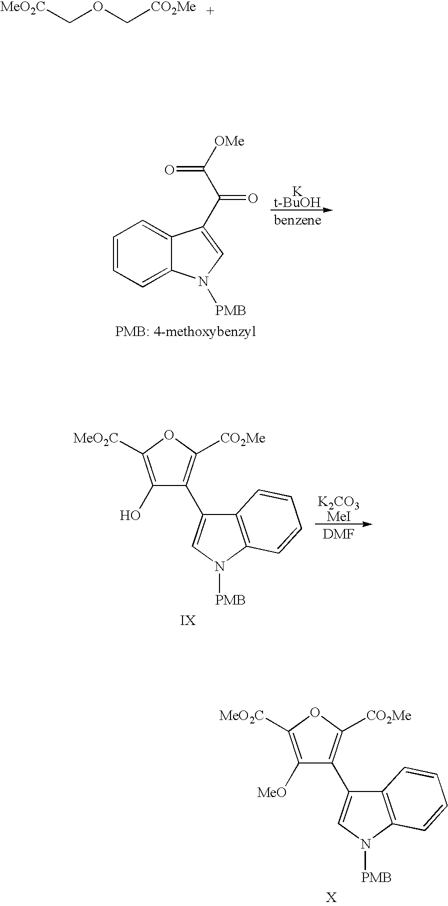

A suspension of potassium t-butoxide was prepared by reacting potassium (20.4 g, 0.78 mol) and t-butanol (38.6 g, 0.52 mol) in benzene (720 mL). The suspension was then kept under reflux. A solution of dimethyl diglycolic acid ester (28.1 g, 0.17 mol) and [1-(4-methoxy-benzyl)-1H-indol-3-yl]-oxo-acetic acid methyl ester (73.8 g, 0.23 mol) in benzene (615 mL) was added dropwise to the above refluxing suspension. After being refluxed under nitrogen for 2.5 hours, the mixture was cooled, diluted with ice water, and acidified with 3 N hydrochloric acid. The resultant mixture was extracted with ethyl acetate. The extracts were combined, washed with water, dried, and concentrated to give Intermediate IX as a crude product (86.0 g).

K₂CO₃ (105.8 g, 0.77 mol) and MeI (81.2 g, 0.572 mol) were added sequentially to a solution of the crude intermediate IX (86.0 g) in DMF (1,550 mL). After being stirred at room temperature for 21 hours, the reaction mixture was filtered from a sintered glass funnel. The filtrate was concentrated under vacuum below 50° C. The residue thus obtained was diluted with dichloromethane and washed with brine and water. The organic layer was collected, dried with anhydrous MgSO₄, and concentrated to give a crude product. The crude product was purified by silica gel column chromatography using 1/4 ethyl acetate-hexane as an eluent to afford Intermediate X (25.6 g, yield in two steps: 33.5%). ¹H-NMR (CDCl₃): 7.54 (d, J=7.9, 1H), 7.48 (s, 1H), 7.34 (d, J=8.1, 1H), 7.22 (t, J=7.6, 1H), 7.15 (t, J=8.6, 3H), 6.85 (d, J=8.6, 1H), 5.32 (s, 1H), 3.97 (s, 3H), 3.78 (m, 9H). LC/MS (M⁺+1): 450.0.

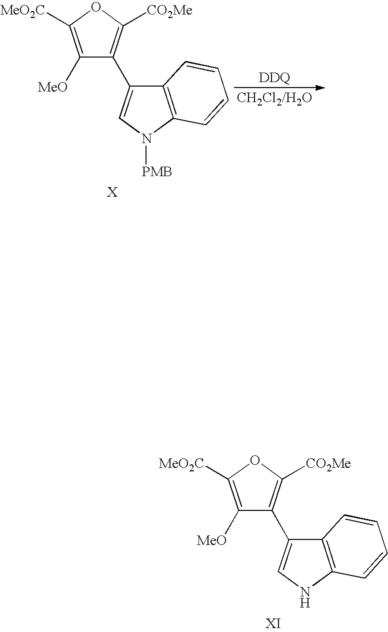

DDQ (25.9 g, 0.11 mol) was added to a solution of intermediate X (25.6 g, 0.057 mol) in CH₂Cl₂ (770 mL) and H₂O (43 ml). After the reaction mixture was stirred at room temperature for 21 hours, it was placed on a water-bath. 5% Na₂CO₃ was then added. The resultant mixture was diluted with dichloromethane, and washed with brine and water. The organic layer was collected, dried with anhydrous MgSO₄, and concentrated to give a crude product. The crude product was purified by silica gel column chromatography using 1/4 ethyl acetate-hexane as an eluent to afford Intermediate XI (11.3 g, 60%). ¹H-NMR (CDCl₃): 8.52 (s, 1H), 7.54 (m, 2H), 7.41 (d, J=8.1, 1H), 7.24 (t, J=7.6, 1H), 7.16 (t, J=7.6, 1H), 3.97 (d, J=4.3, 3H), 3.82 (s, 3H), 3.77 (t, J=2.9, 3H). LC/MS (M⁺+1): 330.3.

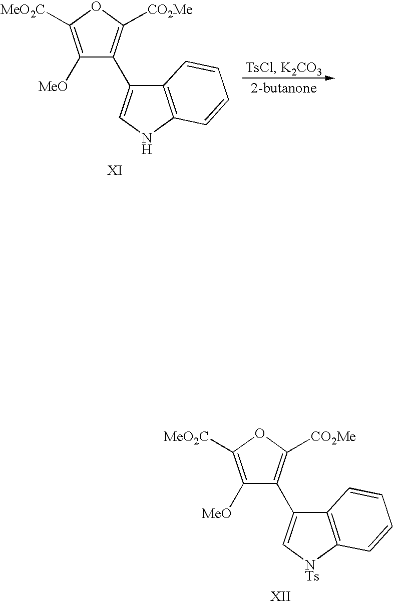

Intermediate XI (15.10 g, 0.046 mol) was mixed with TsCl (17.48 g, 0.092 mol) and K₂CO₃ (19.00 g, 0.137 mol) in 2-butanone (460 ml). After the mixture was refluxed under nitrogen for 2 hours, additional amounts of TsCl (8.7 g, 0.046 mol) and K₂CO₃ (9.50 g, 0.069 mol) were added. The resultant mixture was then refluxed overnight. The reaction mixture was filtered from a sintered glass funnel. The filtrate was concentrated under vacuum below 40° C., and the residue treated with methanol to form a suspension. The suspension was again filtered from a sintered glass funnel. The precipitate was collected and dried to afford Intermediate XII (20.4 g, 91.9%). ¹H-NMR (CDCl₃): 8.00 (d, J=8.3 Hz, 1H), 7.84 (m, 3H), 7.39 (d, J=8.3, 1H), 7.34 (t, J=8.3, 1H), 7.24 (m, 3H), 3.97 (s, 3H), 3.79 (s, 3H), 3.76 (s, 3H). LC/MS (M⁺+1): 484.5.

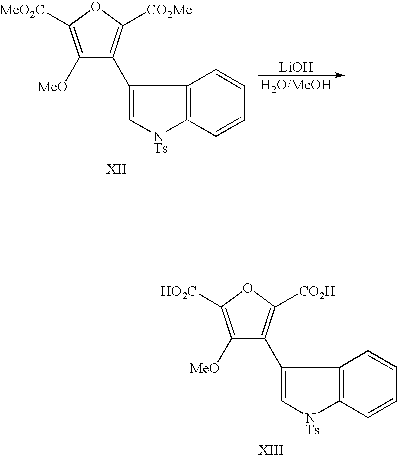

Lithium hydroxide (4.3 g, 177.7 mmol, 700 mol % vs Intermediate XII) was added at 25° C. to a solution of Intermediate XII (12.3 g, 25.4 mmol) in methanol (280 mL) and H₂O (23 mL). The resultant solution was stirred at 25° C. for 5 hours, acidified with 10% sulfuric acid, and concentrated. The residue was diluted with ethyl acetate, and washed with water and brine, dried, and concentrated to give Intermediate XIII (99.3%). ¹H NMR (⁶d-DMSO): 8.00 (s, 1H), 7.96 (d, J=8.3, 1H), 7.89 (d, J=8.0, 2H), 7.38 (m, 4H), 7.27 (t, J=7.5, 1H), 3.74(s, 3H), 2.32(s, 3H). LC/MS (M⁺+1): 456.0.

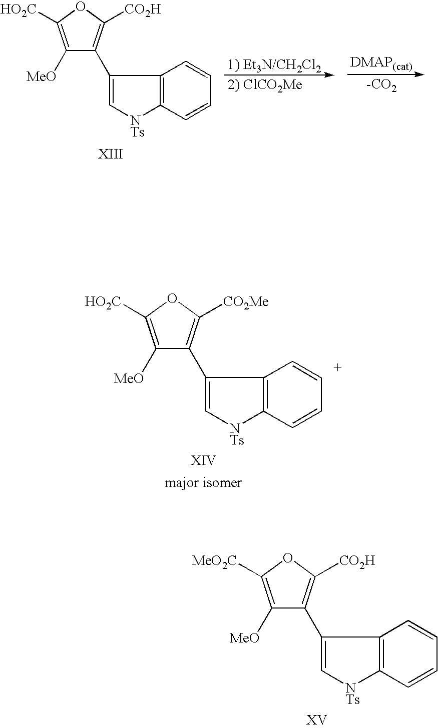

A solution of methyl chloroformate (2.1 g, 22 mmol, 100 mol % vs Intermediate XIII) in dichloromethane (30 mL) was added to a solution of Intermediate XIII (100.0 g, 22.0 mmol) and triethylamine (2.34 g, 23.1 mmol, 105 mole % vs Intermediate XIII) in dichloromethane (100 mL) at 0° C. After the solution was stirred at 0° C. for 60 minutes, DMAP (322 mg) was added. The resulting solution was stirred at 0° C. for 1 hour and at room temperature for additional 18 hours. It was then acidified with 10% sulfuric acid and concentrated. The residue was diluted with ethyl acetate, and washed with water and brine, dried, and concentrated. Intermediates XIV and XV were isolated from the mixture by silica gel column chromatography using 1/2 ethyl acetate/hexane through 100% ethyl acetate to 3/7 (v/v) methanol/ethyl acetate as an eluent.

Intermediate XIV (43.6%): ¹H NMR (⁶d-DMSO): 8.02 (s, 1H), 7.98 (d, J=8.5, 1H), 7.91 (d, J=8.4, 2H), 7.40 (m, 4H), 7.27 (t, J=7.6, 1H), 3.74 (s, 3H), 3.68 (s, 3H), 2.33 (s, 3H). LC/MS: 470.0 (M⁺+1).

Intermediate XV (2.3%): ¹H NMR (⁶d-DMSO): 7.90 (m, 4H), 7.37 (d, J=8.1, 3H), 7.31 (t, J=7.7, 1H), 7.22 (t, J=7.5, 1H), 3.83 (s, 3H), 3.67 (s, 3H), 2.30 (s, 3H).

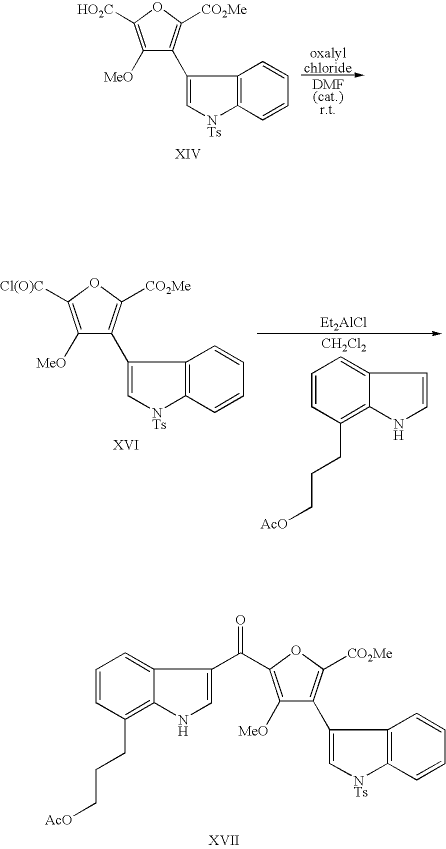

Intermediate XVII was prepared by a method similar to the preparation of Intermediate VII described in Example 1, except that the acyl chloride formation step was carried out at room temperature in oxalyl chloride containing a catalytic amount of DMF. ¹H NMR (⁶d-acetone): 8.66 (d, J=3.2 Hz, 1H), 8.39 (d, J=8.0 Hz, 1H), 8.06 (d, J=8.4 Hz, 1H), 7.99 (s, 1H), 7.95 (d, J=8.3 Hz, 2H), 7.51 (d, J=8.0 Hz, 1H), 7.42 (d, J=8.3 Hz, 2H), 7.39 (m, 1H), 7.29 (m, 1H), 7.23 (m, 1H), 7.17 (m, 1H), 4.12 (t, J=6.6 Hz, 2H), 3.91 (s, 3H), 3.76 (s, 3H), 3.06 (t, J=7.5 Hz, 2H), 2.36 (s, 3H), 2.13 (m, 2H).

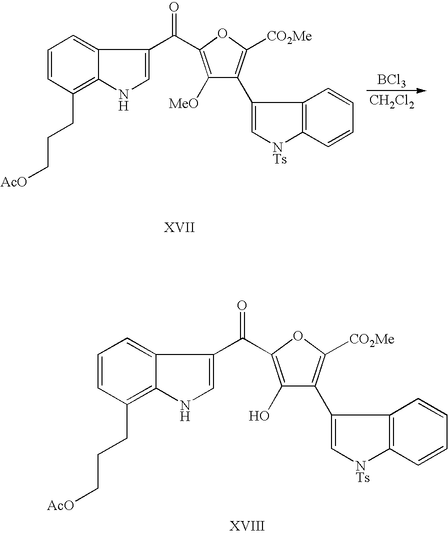

Intermediate XVIII was prepared by the same method as the preparation of Intermeidate VIII described in Example 1. ¹H NMR (⁶d-acetone): 8.80 (d, J=3.2 Hz, 1H), 8.37 (d, J=8.0 Hz, 1H), 8.11 (s, 1H), 8.09 (m, 1H), 8.00 (d, J=8.3 Hz, 2H), 7.65 (d, J=7.9 Hz, 1H), 7.45 (d, J=8.3 Hz, 2H), 7.43 (m, 1H), 7.32 (m, 1H), 7.29 (m, 1H), 7.23 (d, J=7.2 Hz, 1H), 4.15 (t, J=6.6 Hz, 2H), 3.86 (s, 3H), 3.10 (t, J=7.9 Hz, 2H), 2.39 (s, 3H), 2.11 (m, 2H).

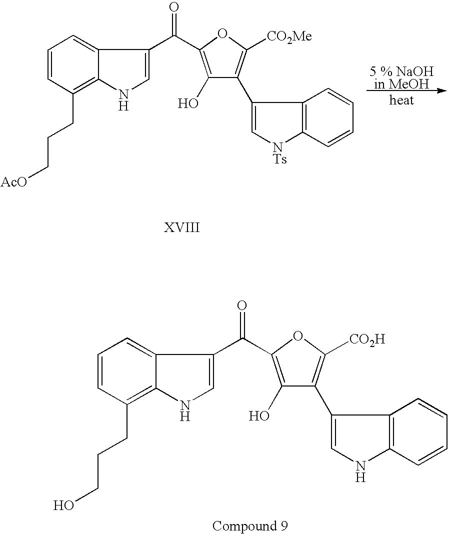

Compound 9 was prepared by a method similar to the preparation of compound 1 described in Example 1, except that the hydrolysis was carried out in a refluxing solution MeOH solution containing 5% NaOH. ¹H-NMR (⁶d-acetone): 8.85 (br, s, 1H), 8.32 (d, J=7.8 Hz, 1H), 8.00 (br s, 1H), 7.78 (m, 1H), 7.67 (m, 1H), 7.60 (m, 1H), 7.48 (m, 1H), 7.25 (m, 1H), 7.18 (m, 2H), 7.10 (m, 1H), 3.65 (t, J=6.4 Hz, 2H), 3.01 (t, J=7.7 Hz, 2H), 2.11 (m, 2H). LC/MS (negative mode) (M⁺−1): 443.2.

### EXAMPLES 3-173

### Preparation of Compounds 2-8 and 10-173

Compounds 2-8 and 10-173 were prepared in a manner similar to that described in Example 1 or Example 2 by using corresponding starting materials.

Spectroscopic data on compounds 8, 20, 21, 26, 30, 61, 85, 114, and 170-173 are listed below:

Compound 8: ¹H-NMR (⁶d-acetone): 11.45 (br s, 1H), 8.81(d, J=3.3 Hz, 1H), 8.37 (d, J=7.6 Hz, 1H), 7.70 (m, 2H), 7.45 (m, 3H), 7.25 (m, 2H), 3.26 (t, J=7.6 Hz, 2H), 2.79 (t, J=7.6 Hz, 2H). LC/MS (M⁺+1): 420.0.

Compound 20: ¹H-NMR (⁶d-acetone): 11.40 (br s, 1H), 8.78 (d, J=3.4 Hz, 1H), 8.34 (d, J=7.9 Hz, 1H), 7.70 (m, 2H), 7.46 (m, 3H), 7.25 (m, 1H), 7.19 (d, J=7.3 Hz, 1H), 3.64 (t, J=6.4 Hz, 2H), 3.01 (t, J=7.7 Hz, 2H), 1.85 (m, 2H), 1.64 (m, 2H). LC/MS (M⁺+1): 420.0.

Compound 21: ¹H-NMR (⁶d-acetone): 8.90 (br s, 1H), 8.60 (br s, 1H), 8.35 (d, J=8.0 Hz, 1H), 7.80 (m, 1H), 7.68 (d, J=8.0 Hz, 1H), 7.48 (d, J=8.0 Hz, 1H), 7.36 (m, 1H), 7.24 (m, 1H), 7.16 (m, 2H), 7.08 (m, 1H), 3.64 (t, J=6.5 Hz, 2H), 3.01 (t, J=7.5 Hz, 2H), 1.85 (m, 2H), 1.64 (m, 2H). LC/MS (negative mode) (M⁺−1): 457.2.

Compound 26: ¹H-NMR (⁶d-acetone): 8.55 (br s, 1H), 8.42 (d, J=3.3 Hz, 1H), 8.21 (d, J=7.9 Hz, 1H), 7.63 (m, 2H), 7.39 (m, 3H), 7.18 (m, 1H), 7.12 (d, J=7.2 Hz, 1H), 3.95 (s, 3H), 3.60 (s, 3H), 2.97 (t, J=7.3 Hz, 2H), 2.35 (t, J=7.5 Hz, 2H), 1.76 (m, 2H), 1.70 (m, 2H).

Compound 30: ¹H-NMR (⁶d-acetone): ¹H-NMR (⁶d-acetone): 11.4 (br s, 1H), 10.7 (br s, 1H), 8.84 (d, J=3.2 Hz, 1H), 8.38 (d, J=7.9 Hz, 1H), 7.79 (d, J=2.5 Hz, 1H), 7.69 (d, J=8.0 Hz, 1H), 7.51 (d, J=8.0 Hz, 1H), 7.27 (m, 1H), 7.19 (m, 2H), 7.11 (m, 1H), 3.58 (t, J=6.2 Hz, 2H), 3.01 (t, J=7.8 Hz, 2H), 1.82 (t, J=7.4 Hz, 2H), 1.61 (t, J=7.2 Hz, 2H), 1.54 (m, 2H). LC/MS (M⁺+1): 473.0.

Compound 61: ¹H-NMR (⁶d-acetone): 8.79 (d, J=3.1 Hz, 1H), 8.34 (d, J=7.8 Hz, 1H), 7.63 (d, J=8.1 Hz, 2H), 7.33 (d, J=8.1 Hz, 2H), 7.25 (m, 1H), 7.17 (d, J=8.0 Hz, 1H), 3.64 (t, J=6.2 Hz, 2H), 3.06 (t, J=7.5 Hz, 3H), 2.60 (m, 1H), 1.10-2.10 (m, 12H). LC/MS (M⁺+1): 488.0.

Compound 85: ¹H-NMR (⁶d-acetone): 13.1 (s, 1H), 11.51 (br s, 1H), 8.60 (d, J=3.1 Hz, 1H), 8.28 (d, J=7.9 Hz, 1H), 7.50 (m, 2H), 7.38 (m, 3H), 7.25 (m, 1H), 7.19 (d, J=7.0 Hz, 1H), 3.64 (t, J=6.2 Hz, 2H), 3.06 (t, J=7.5 Hz, 2H), 1.96 (m, 2H). LC/MS (M⁺+1): 422.0.

Compound 114: ¹H-NMR (⁶d-DMSO): 13.2 (br s, 1H, ArOH), 12.4 (br s, 1H), 11.2 (br s, 1H), 8.41 (s, 1H), 8.13 (d, J=7.9 Hz, 1H), 7.52 (s, 1H), 7.41 (m, 2H), 7.17 (m,1H), 7.08 (m, 2H), 6.96 (m, 1H), 3.39 (t, J=7.1 Hz, 2H), 2.91 (t, J=7.6 Hz, 2H), 1.68 (m, 2H), 1.48 (m, 2H), 1.40 (m, 2H). LC/MS (M⁺+1): 489.0.

Compound 170: ¹H-NMR (⁶d-acetone): 11.5 (br s, 1H), 10.7 (br s, 1H), 8.85 (d, J=3.1 Hz, 1H), 8.39 (d, J=7.6 Hz, 1H), 7.77 (d, J=2.6 Hz, 1H), 7.67 (d, J=8.0 Hz, 1H), 7.50 (d, J=8.1 Hz, 1H), 7.26 (m, 2H), 7.17 (m, 1H), 7.09 (m, 1H), 3.26 (m, 4H). LC/MS (M⁺): 458.5.

Compound 171: ¹H-NMR (⁴d-MeOH): 8.86 (br s, 1H), 8.28 (d, J=7.8 Hz, 1H), 7.59 (d, J=8.7, 2H), 7.39 (d, J=8.1 1H), 7.17 (m, 1H), 7.11 (m, 1H), 7.04 (m, 2H), 2.90 (br s, 2H), 1.73 (br s, 2H), 1.39 (br s, 4H), 0.90 (m, 3H). LC/MS (M⁺+1): 457.0.

Compound 172: ¹H-NMR (⁶d-acetone): 11.44 (br s, 1H), 10.66(br s, 1H), 8.83 (d, J=3.0 Hz, 1H), 8.37 (d, J=7.8 Hz, 1H), 7.77 (d, J=2.4 Hz, 1H), 7.67 (d, J=7.8 Hz, 1H), 7.50 (d, J=8.1 Hz, 1H), 7.26 (m, 1H), 7.18 (m, 2H), 7.09 (m, 1H), 3.02 (t, J=7.5 Hz, 2H), 2.38 (t, J=7.5 Hz, 2H), 1.92 (d, J=2.1 Hz, 2H), 1.74 (d, J=7.6 Hz, 2H). LC/MS (negative mode) (M⁺−1): 486.5.

Compound 173: ¹H-NMR (⁶d-acetone): 11.52 (br s, 1H), 10.68 (br s, 1H), 8.84 (d, J=3.1 Hz, 1H), 8.38 (d, J=7.8 Hz, 1H), 7.79 (d, J=2.6 Hz, 1H), 7.68 (d, J=8.0 Hz, 1H), 7.51 (d, J=8.1 Hz, 1H), 7.27 (m, 1H), 7.19 (m, 2H), 7.11 (m, 1H), 3.64 (m, 4H), 3.01 (t, J=7.9 Hz, 2H), 1.87 (m, 2H), 1.75 (m, 1H), 1.50 (m, 2H). LC/MS (M⁺+1): 503.0.

### EXAMPLE 174

### Cell-Based Assay for Insulin Receptor Tyrosine Phosphorylation

CHO.IR cells overexpressing human insulin receptor were obtained from Stanford University. Approximately 1.5×10⁵ cells were seeded in each well of a 96-well plate containing Hams F12 medium plus 10% fetal calf serum, fungizone, penicillin, and streptomycin. The cells were incubated at 37° C. for approximately 24 hours, allowing cells to reach confluency. The cells were then washed with phosphate buffered saline (PBS) three times and then incubated in serum-free medium at 37° C. for 2 hours. Insulin or test compounds were added to the wells and the cells were incubated for an additional 20 minutes at 37° C. The cells were washed with PBS three times and then lysed in 60 μL/well of a lysis buffer containing 50 mM HEPES, pH 7.4, 1% Triton X-100, 5 mM EDTA, 5 mM EGTA, 20 mM sodium pyrophosphate, 1 mM sodium vanadate, 20 mM sodium fluoride, 1 mg/mL Bacitracin, 150 mM sodium chloride, 2 μg/mL Aprotonin, and 1 mM PMSF. Lysates were transferred to a second 96-well plate pre-coated with monoclonal anti-insulin receptor antibody (0.2 μg/50 μL/well, in 20 mM NaHCO₃, pH 9.6). The second plate was incubated at 4° C. for 16 hours to immunoabsorb insulin receptors.

The second plate was washed and probed with monoclonal antiphosphotyrosine antibody conjugated with alkaline phosphatase (Transduction Laboratories, Lexington, Ky.) at 4° C. for 5 hours. After removing the unbound antibody, a chromogenic substrate of alkaline phosphotase was added to the wells. The level of tyrosine phosphorylation on insulin receptors was determined by detecting absorption signals at 405 nm with a microtiter plate reader.

### EXAMPLE 175

### Cell-Based Assay for Insulin Receptor Tyrosine Kinase Activity

CHO.IR cells were cultured at approximately 1.5×10⁵ cells/well (in a 96-well plate) in a Hams F12 medium supplemented with 10% fetal calf serum, fungizone, penicillin, and streptomycin. The cells were incubated at 37° C. for 24 hours, allowing cells to reach confluency. The cells were then washed with PBS three times and then incubated in serum-free medium at 37° C. for 2 hours. Insulin or test compounds were added to the cells and incubated for an additional 20 minutes at 37° C. The cells were washed three times with PBS and lysed in the same 60 μL lysis buffer as mentioned in Example 174. Lysates were transferred to a second 96-well plate pre-coated with monoclonal anti-insulin receptor antibody (under the same conditions as those mentioned in Example 174). Lysates were incubated at 4° C. for 16 hours to immunoabsorb insulin receptors.

20 μL of a kinase reaction mixture (50 mM Hepes, pH 7.6, 150 mM NaCl, 5 mM MgCl₂, 5 mM MnCl₂, 0.1% Triton x-100, 1 mg/mL poly(Glu:Tyr)(4:1), 2 μCi of carrier-free [γ-³²P]ATP) was added to each well. After reacting at 25° C. for 40 minutes, the reaction mixture was transferred to a Multiscreen pH plate (Millipore, Billerica, Mass.) and unbound ATP was washed away. The insulin receptor tyrosine kinase activity was determined by detecting the radioactivities associated with the wells using a Topcount scintillation counter (Perkin-Elmer, Wellesley, Mass.).

The results show that cells treated with 100 μM of compounds 9, 21, 30, 114, and 170-173 exhibited a 12%-53% reduction in tyrosine kinase activity. Cells treated with between 1 and 100 μM of these compounds showed dose-dependent responses.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I): 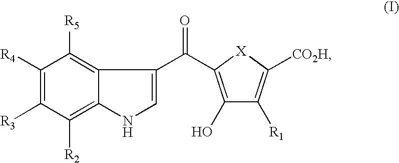
wherein
X is O;
R₁ is H, ORₐ, C₁-C₁₀ alkyl, C₃-C₂₀ cycloalkyl, C₃-C₂₀ heterocycloalkyl, aryl, 5-membered heteroaryl, 6-membered heteroaryl, or fused heteroaryl optionally substituted with C₁-C₄ alkyl, C₃-C₇ cycloalkyl, C₃-C₇ heterocycloalkyl, aryl, heteroaryl, halogen, or ORₐ;
R₂ is C₁-C₁₀ alkyl optionally substituted with OR_{b}, COOR_{b}, C(O)NR_{b}R_{c}, or NR_{b}—C(O)R_{c};
each of R₃, R₄, and R₅, independently, is H, OR_{d}, halogen, C₁-C₁₀ alkyl, C₃-C₂₀ cycloalkyl, C₃-C₂₀ heterocycloalkyl, aryl, or heteroaryl;
in which each of Rₐ, R_{b}, R_{c}, and R_{d}, independently, is H, C₁-C₁₀ alkyl, C₃-C₂₀ cycloalkyl, C₃-C₂₀ heterocycloalkyl, aryl, or heteroaryl.

2. The compound of claim 1, wherein R₁ is 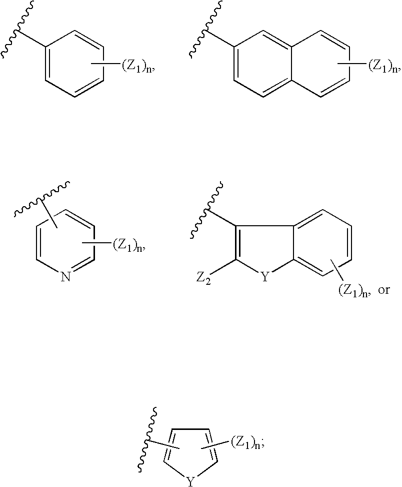
in which Y is O, S, N(R); n is 0-3; and each of Z₁ and Z₂, independently, is C₁-C₄ alkyl, C₃-C₇ cycloalkyl, C₃-C₇ heterocycloalkyl, aryl, heteroaryl, halogen, or OR′; each of R and R′, independently, being C₁-C₄ alkyl, C₃-C₇ cycloalkyl, C₃-C₇ heterocycloalkyl, aryl, or heteroaryl.

3. The compound of claim 2, wherein R₁ is 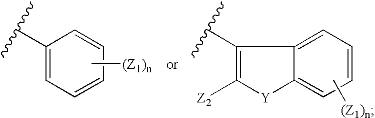
in which Y is NH; n is 0 or 1; and each of Z₁ and Z₂, independently, is C₁-C₄ alkyl, C₃-C₇ cycloalkyl, halogen, or OR′.

4. The compound of claim 3, wherein R₂ is C₁-C₁₀ alkyl optionally substituted with OR_{b} or COOR_{b}.

5. The compound of claim 4, wherein the compound is one of compounds 1-84 and 169-173.

6. The compound of claim 1, wherein R₂ is C₁-C₁₀ alkyl optionally substituted with OR_{b} or COOR_{b}.

7. A compound of formula (I): 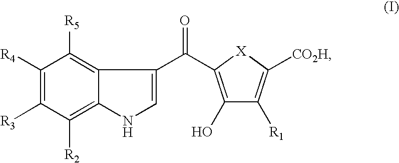
wherein
X is S;
R₁ is H, C₁-C₁₀ alkyl, C₃-C₂₀ cycloalkyl, C₃-C₂₀ heterocycloalkyl, aryl, heteroaryl, or ORₐ;
R₂ is unsubstituted C₁-C₁₀ alkyl or C₁-C₁₀ alkyl substituted with OR_{b}, COOR_{b}, C(O)NR_{b}R_{c}, or NR_{b}—C(O)R_{c};
each of R₃, R₄, and R₅, independently, is H, OR_{d}, halogen, C₁-C₁₀ alkyl, C₃-C₂₀ cycloalkyl, C₃-C₂₀ heterocycloalkyl, aryl, or heteroaryl;
in which each of Rₐ, R_{b}, R_{c}, and R_{d}, independently, is H, C₁-C₁₀ alkyl, C₃-C₂₀ cycloalkyl, C₃-C₂₀ heterocycloalkyl, aryl, or heteroaryl.

8. The compound of claim 7, wherein R₁ is 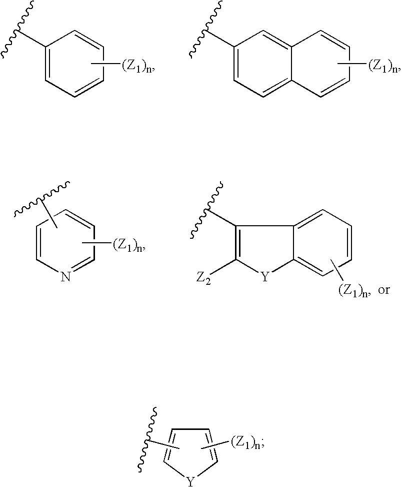
in which Y is O, S, N(R); n is 0-3; and each of Z₁ and Z₂, independently, is C₁-C₄ alkyl, C₃-C₇ cycloalkyl, C₃-C₇ heterocycloalkyl, aryl, heteroaryl, halogen, or OR′; each of R and R′, independently, being C₁-C₄ alkyl, C₃-C₇ cycloalkyl, C₃-C₇ heterocycloalkyl, aryl, or heteroaryl.

9. The compound of claim 8, wherein R₁ is 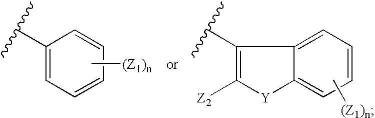
in which Y is NH; n is 0 or 1; and each of Z₁ and Z₂, independently, is C₁-C₄ alkyl, C₃-C₇ cycloalkyl, halogen, or OR′.

10. The compound of claim 9, wherein R₂ is C₁-C₁₀ alkyl substituted with OR_{b} or COOR_{b}.

11. The compound of claim 10, wherein the compound is one of compounds 1-84.

12. The compound of claim 7, wherein R₂ is C₁-C₁₀ alkyl substituted with OR_{b} or COOR_{b}.

13. A method of treating diabetes mellitus, comprising administering to a subject in need thereof an effective amount of a compound of formula (I): 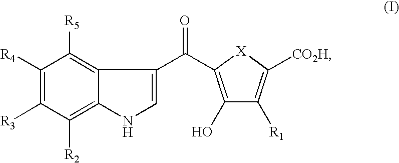
wherein
X is O;
R₁ is H, ORₐ, C₁-C₁₀ alkyl, C₃-C₂₀ cycloalkyl, C₃-C₂₀ heterocycloalkyl, aryl, 5-membered heteroaryl, 6-membered heteroaryl, or fused heteroaryl optionally substituted with C₁-C₄ alkyl, C₃-C₇ cycloalkyl, C₃-C₇ heterocycloalkyl, aryl, heteroaryl, halogen, or ORₐ;
R₂ is C₁-C₁₀ alkyl optionally substituted with OR_{b}, COOR_{b}, C(O)NR_{b}R_{c}, or NR_{b}—C(O)R_{c};
each of R₃, R₄, and R₅, independently, is H, OR_{d}, halogen, C₁-C₁₀ alkyl, C₃-C₂₀ cycloalkyl, C₃-C₂₀ heterocycloalkyl, aryl, or heteroaryl;
in which each of Rₐ, R_{b}, R_{c}, and R_{d}, independently, is H, C₁-C₁₀ alkyl, C₃-C₂₀ cycloalkyl, C₃-C₂₀ heterocycloalkyl, aryl, or heteroaryl.

14. The method of claim 13, wherein R₁ is 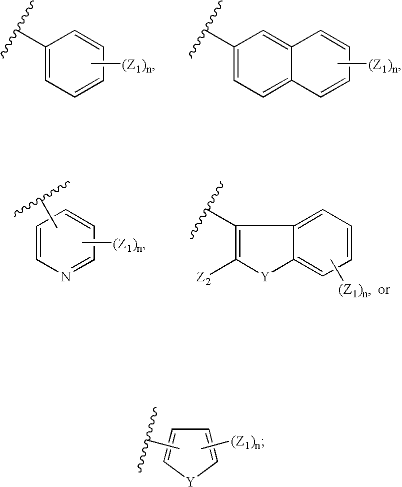
in which Y is O, S, N(R); n is 0-3; and each of Z₁ and Z₂, independently, is C₁-C₄ alkyl, C₃-C₇ cycloalkyl, C₃-C₇ heterocycloalkyl, aryl, heteroaryl, halogen, or OR′; each of R and R′, independently, being C₁-C₄ alkyl, C₃-C₇ cycloalkyl, C₃-C₇ heterocycloalkyl, aryl, or heteroaryl.

15. The method of claim 14, wherein R₁ is 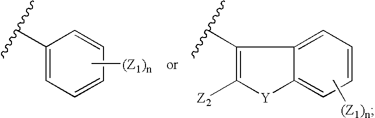
in which Y is NH; n is 0 or 1; and each of Z₁ and Z₂, independently, is C₁-C₄ alkyl, C₃-C₇ cycloalkyl, halogen, or OR′.

16. The method of claim 15, wherein R₂ is C₁-C₁₀ alkyl optionally substituted with OR_{b} or COOR_{b}.

17. The method of claim 13, wherein R₂ is C₁-C₁₀ alkyl optionally substituted with OR_{b} or COOR_{b}.

18. A method of treating diabetes mellitus, comprising administering to a subject in need thereof an effective amount of a compound of formula (I): 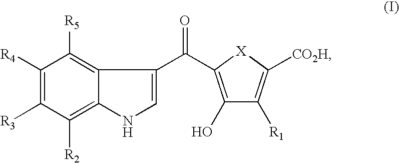
wherein
X is S;
R₁ is H, C₁-C₁₀ alkyl, C₃-C₂₀ cycloalkyl, C₃-C₂₀ heterocycloalkyl, aryl, heteroaryl, or ORₐ;
R₂ is unsubstituted C₁-C₁₀ alkyl or C₁-C₁₀ alkyl substituted with OR_{b}, COOR_{b}, C(O)NR_{b}R_{c}, or NR_{b}—C(O)R_{c};
each of R₃, R₄, and R₅, independently, is H, OR_{d}, halogen, C₁-C₁₀ alkyl, C₃-C₂₀ cycloalkyl, C₃-C₂₀ heterocycloalkyl, aryl, or heteroaryl;
in which each of Rₐ, R_{b}, R_{c}, and R_{d}, independently, is H, C₁-C₁₀ alkyl, C₃-C₂₀ cycloalkyl, C₃-C₂₀ heterocycloalkyl, aryl, or heteroaryl.

19. The method of claim 18, wherein R₁ is 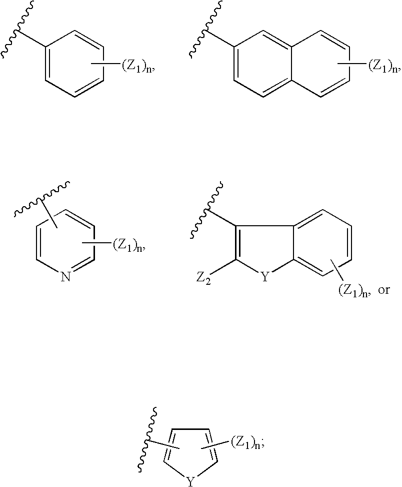
in which Y is O, S, N(R); n is 0-3; and each of Z₁ and Z₂, independently, is C₁-C₄ alkyl, C₃-C₇ cycloalkyl, C₃-C₇ heterocycloalkyl, aryl, heteroaryl, halogen, or OR′; each of R and R′, independently, being C₁-C₄ alkyl, C₃-C₇ cycloalkyl, C₃-C₇ heterocycloalkyl, aryl, or heteroaryl.

20. The method of claim 19, wherein R₁ is 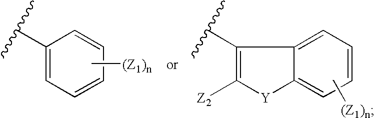
in which Y is NH; n is 0 or 1; and each of Z₁ and Z₂, independently, is C₁-C₄ alkyl, C₃-C₇ cycloalkyl, halogen, or OR′.

21. The method of claim 20, wherein R₂ is C₁-C₁₀ alkyl substituted with OR_{b} or COOR_{b}.

22. The method of claim 18, wherein R₂ is C₁-C₁₀ alkyl substituted with OR_{b} or COOR_{b}.

23. A compound of formula (II): 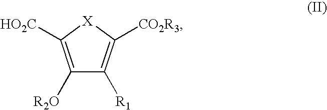
wherein
X is O or S;
R₁ is H, C₁-C₁₀ alkyl, C₃-C₂₀ cycloalkyl, C₃-C₂₀ heterocycloalkyl, aryl, heteroaryl, or ORₐ;
R₂ is a hydroxyl protecting group; and
R₃ is a carboxyl protecting group.

24. The compound of claim 23, wherein R₁ is 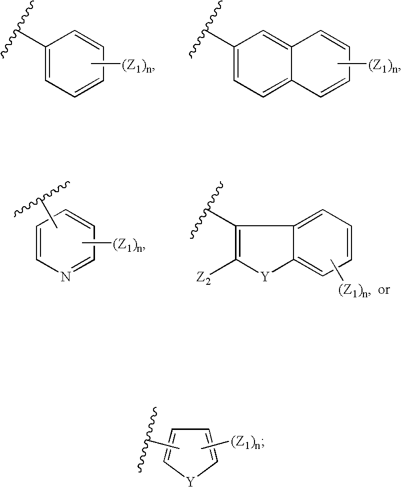
in which Y is O, S, N(R); n is 0-3; and each of Z₁ and Z₂, independently, is C₁-C₄ alkyl, C₃-C₇ cycloalkyl, C₃-C₇ heterocycloalkyl, aryl, heteroaryl, halogen, or OR′; each of R and R′, independently, being C₁-C₄ alkyl, C₃-C₇ cycloalkyl, C₃-C₇ heterocycloalkyl, aryl, or heteroaryl.

25. The compound of claim 24, wherein R₁ is 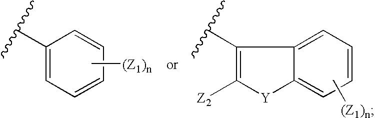
in which Y is NH; n is 0 or 1; and each of Z₁ and Z₂, independently, is C₁-C₄ alkyl, C₃-C₇ cycloalkyl, halogen, or OR′.

26. The compound of claim 25, wherein each of R₂ and R₃, independently, is C₁-C₁₀ alkyl or aryl.

27. The compound of claim 26, wherein R₂ is methyl, methoxymethyl, or benzyl.

28. The compound of claim 26, wherein R₃ is methyl, benzyl, or aryl.

29. The compound of claim 23, wherein each of R₂ and R₃, independently, is C₁-C₁₀ alkyl or aryl.

30. A method of preparing a compound of formula (II): 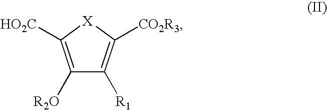
the method comprising
reacting a compound of formula (III): 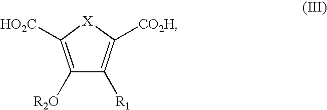
with R₄CO₂R₃ in a basic condition, and then with a tertiary amine; wherein
X is O or S;
R₁ is H, C₁-C₁₀ alkyl, C₃-C₂₀ cycloalkyl, C₃-C₂₀ heterocycloalkyl, aryl, heteroaryl, or ORₐ;
R₂ is a hydroxyl protecting group;
R₃ is a carboxyl protecting group; and
R₄ is halogen.
